(19) United States
(12) Patent Application Publication
(10) Pub. No.: US 2006/0018877 A1
(43) Pub. Date: Jan. 26, 2006

(54) Intradermal delivery of vacccines and therapeutic agents

(76) Inventors: John A. Mikszta, Durham, NC (US); Jason B. Alarcon, Durham, NC (US); Cheryl Dean, Raleigh, NC (US); Andrea Hartley, Hampstead, NC (US)

Correspondence Address: JONES DAY, 222 EAST 41ST ST, NEW YORK, NY, 10017, US

(21) Appl. No.: 11/118,916

(22) Filed: Apr. 29, 2005

Related U.S. Application Data
(63) Continuation-in-part of application No. 10/679,038, filed on Oct. 2, 2003.
Application No. 10/679,038 is a continuation-in-part of application No. 10/185,717, filed on Jul. 1, 2002.
(60) Provisional application No. 60/301,476, filed on Jun. 29, 2001.
(60) Provisional application No. 60/566,629, filed on Apr. 29, 2004.

(51) Int. Cl. A61K 39/145

(52) U.S. Cl. 424/93.1; 604/522; 604/500; 424/209.1

(57) ABSTRACT

The present invention relates to methods and devices for administration of vaccines and therapeutic agents into the intradermal layer of the skin. The methods of the present invention elicit increased humoral and/or cellular response as compared to conventional vaccine delivery methods, e.g., intramuscular route. Furthermore, the methods of the present invention facilitate induction of an immune response by an amount of vaccine which is otherwise insufficient for inducing an immune response when delivered via conventional vaccine routes, e.g., intramuscular route.

This application claims the benefit of Provisional Application Ser. No. 60/566,629, filed Apr. 29, 2004, which is incorporated by reference in its entirety.

### 1. FIELD OF THE INVENTION

The present invention relates to methods and devices for administration of vaccines and therapeutic agents into the intradermal layer of the skin. The methods of the present invention elicit increased humoral and/or cellular response as compared to conventional vaccine delivery methods, e.g., intramuscular route. Furthermore, the methods of the present invention facilitate induction of an immune response by an amount of vaccine which is otherwise insufficient for inducing an immune response when delivered via conventional vaccine routes, e.g., intramuscular route.

### 2. BACKGROUND INFORMATION

The importance of efficiently and safely administering pharmaceutical substances for the purpose of prophylaxis, diagnosis or treatment has long been recognized. The use of conventional needles has long provided one approach for delivering pharmaceutical substances to humans and animals by administration through the skin. Considerable effort has been made to achieve reproducible and efficacious delivery through the skin while improving the ease of injection and reducing patient apprehension and/or pain associated with conventional needles. Furthermore, certain delivery systems eliminate needles entirely, and rely upon chemical mediators or external driving forces such as iontophoretic currents or electroporation or thermal poration or sonophoresis to breach the stratum corneum, the outermost layer of the skin, and deliver substances through the surface of the skin. However, such delivery systems do not reproducibly breach the skin barriers or deliver the pharmaceutical substance to a given depth below the surface of the skin and consequently, clinical results can be variable. Thus, mechanical breach of the stratum corneum such as with needles, is believed to provide the most reproducible method of administration of substances through the surface of the skin, and to provide control and reliability in placement of administered substances.

Approaches for delivering substances beneath the surface of the skin have almost exclusively involved transdermal administration, i.e. delivery of substances through the skin to a site beneath the skin. Transdermal delivery includes subcutaneous, intramuscular or intravenous routes of administration of which, intramuscular (IM) and subcutaneous (SC) injections have been the most commonly used.

Anatomically, the outer surface of the body is made up of two major tissue layers, an outer epidermis and an underlying dermis, which together constitute the skin (for review, see Physiology, Biochemistry, and Molecular Biology of the Skin, Second Edition, L. A. Goldsmith, Ed., Oxford University Press, New York, 1991). The epidermis is subdivided into five layers or strata of a total thickness of between 75 and 150 μm. Beneath the epidermis lies the dermis, which contains two layers, an outermost portion referred to at the papillary dermis and a deeper layer referred to as the reticular dermis. The papillary dermis contains vast microcirculatory blood and lymphatic plexuses. In contrast, the reticular dermis is relatively acellular and avascular and made up of dense collagenous and elastic connective tissue. Beneath the epidermis and dermis is the subcutaneous tissue, also referred to as the hypodermis, which is composed of connective tissue and fatty tissue. Muscle tissue lies beneath the subcutaneous tissue.

As noted above, both the subcutaneous tissue and muscle tissue have been commonly used as sites for administration of pharmaceutical substances. The dermis, however, has rarely been targeted as a site for administration of substances, and this may be due, at least in part, to the difficulty of precise needle placement into the intradermal (ID) space. Furthermore, even though the dermis, in particular, the papillary dermis has been known to have a high degree of vascularity, it has not heretofore been appreciated that one could take advantage of this high degree of vascularity to obtain an improved absorption profile for administered substances compared to subcutaneous administration. This is because small drug molecules are typically rapidly absorbed after administration into the subcutaneous tissue that has been far more easily and predictably targeted than the dermis has been. On the other hand, large molecules such as proteins are typically not well absorbed through the capillary epithelium regardless of the degree of vascularity so that one would not have expected to achieve a significant absorption advantage over subcutaneous administration by the more difficult to achieve intradermal administration even for large molecules.

One approach to administration beneath the surface to the skin and into the region of the intradermal space has been routinely used in the Mantoux tuberculin test. In this procedure, a purified protein derivative is injected at a shallow angle to the skin surface using a 27 or 30 gauge needle and standard syringe (Flynn et al., Chest 106: 1463-5, 1994). The Mantoux technique involves inserting the needle into the skin laterally, then “snaking” the needle further into the ID tissue. The technique is known to be quite difficult to perform and requires specialized training. A degree of imprecision in placement of the injection results in a significant number of false negative test results. Moreover, the test involves a localized injection to elicit a response at the site of injection and the Mantoux approach has not led to the use of intradermal injection for systemic administration of substances. Another group reported on what was described as an intradermal drug delivery device (U.S. Pat. No. 5,997,501). Injection was indicated to be at a slow rate and the injection site was intended to be in some region below the epidermis, i.e., the interface between the epidermis and the dermis or the interior of the dermis or subcutaneous tissue. This reference, however, provided no teachings that would suggest a selective administration into the dermis nor did the reference suggest that vaccines or gene therapeutic agents might be delivered in this manner. To date, numerous therapeutic proteins and small molecular weight compounds have been delivered intradermally and used to effectively elicit a pharmacologically beneficial response. Most of these compounds (e.g., insulin, Neupogen, hGH, calcitonin) have been hormonal proteins, not engineered receptors or antibodies. To date all administered proteins have exhibited several effects associated with ID administration, including more rapid onset of uptake and distribution (vs. SC) and in some case increased bioavailability.

Dermal tissue represents an attractive target site for delivery of vaccines and gene therapeutic agents. In the case of vaccines (both genetic and conventional), the skin is an attractive delivery site due to the high concentration of antigen presenting cells (APC) and APC precursors found within this tissue, in particular the epidermal Langerhan's cells and dermal dendritic cells. Several gene therapeutic agents are designed for the treatment of skin disorders, skin diseases and skin cancer. In such cases, direct delivery of the therapeutic agent to the affected skin tissue is desirable. In addition, skin cells are an attractive target for gene therapeutic agents, of which the encoded protein or proteins are active at sites distant from the skin. In such cases, skin cells (e.g., keratinocytes) can function as “bioreactors” producing a therapeutic protein that can be rapidly absorbed into the systemic circulation via the papillary dermis. In other cases, direct access of the vaccine or therapeutic agent to the systemic circulation is desirable for the treatment of disorders distant from the skin. In such cases, systemic distribution can be accomplished through the papillary dermis.

However, as discussed above, intradermal (ID) injection using standard needles and syringes is technically very difficult to perform and is painful. The prior art contains several references to ID delivery of both DNA-based and conventional vaccines and therapeutic agents, however results have been conflicting, at least in part due to difficulties in accurately targeting the ID tissue with existing techniques.

Virtually all of the human vaccines currently on the market are administered via the IM or SC routes. Of the 32 vaccines marketed by the 4 major global vaccine producers in the year 2001 (Aventis-Pasteur, GlaxoSmithKIine, Merck, Wyeth), only 2 are approved for ID use (2001 Physicians Desk Reference). In fact, the product inserts for 6 of these 32 vaccines specifically states not to use the ID route. This is despite the various published pre-clinical and early clinical studies suggesting that ID delivery can improve vaccines by inducing a stronger immune response than via IM or SC injection or by inducing a comparable immune response at a reduced dose relative to that which is given IM or SC (Playford, E. G. et al., 2002, Infect. Control Hosp. Epidemiol. 23:87; Kerr, C. 2001, Trends Microbiol. 9:415; Rahman, F. et al., 2000, Hepatology 31:521; Carlsson, U. et al., 1996, Scan J. Infect. Dis. 28:435; Propst, T. et al., 1998, Amer. J. Kidney Dis. 32:1041; Nagafuchi, S. et al., 1998, Rev Med Virol., 8:97; Henderson, E. A., et al., 2000. Infect. Control Hosp Epidemiol. 21:264). Although improvements in vaccine efficacy following ID delivery have been noted in some cases, others have failed to observe such advantages (Crowe, 1965, Am. J. Med. Tech. 31:387-396; Letter to British Medical Journal 29/10/77, p. 1152; Brown et al., 1977, J. Infect. Dis. 136:466-471; Herbert & Larke, 1979, J. Infect. Dis. 140:234-238; Ropac et al. Periodicum Biologorum 2001, 103:39-43).

A major factor that has precluded the widespread use of the ID delivery route and has contributed to the conflicting results described above is the lack of suitable devices to accomplish reproducible delivery to the epidermal and dermal skin layers. Standard needles commonly used to inject vaccines are too large to accurately target these tissue layers when inserted into the skin. The most common method of delivery is through Mantoux-style injection using a standard needle and syringe. This technique is difficult to perform, unreliable and painful to the subject. Thus, there is a need for devices and methods that will enable efficient, accurate and reproducible delivery of vaccines and gene therapeutic agents to the intradermal layer of skin.

### 3. SUMMARY OF THE INVENTION

The present invention improves the clinical utility of ID delivery of vaccines and gene therapeutic agents to humans or animals. The methods employ devices to directly target the intradermal space and to deliver substances to the intradermal space as a bolus or by infusion. It has been discovered that the placement of the substance within the dermis provides for efficacious and/or improved responsiveness to vaccines and gene therapeutic agents. The device is so designed as to prevent leakage of the substance from the skin and improve adsorption or cellular uptake within the intradermal space. The immunological response to a vaccine delivered according to the methods of the invention has been found to be improved over conventional IM delivery of the vaccine indicating that intradermal administration according to the methods of the invention will in many cases improve clinical results in addition to the other advantages of intradermal delivery.

The present inventors have discovered that the methods of vaccine delivery of the present invention elicit an increased humoral and/or cellular immune response compared to conventional methods of vaccine delivery, e.g., intramuscular delivery. Furthermore, the methods of the present invention enable a reduced dose of vaccine to elicit a humoral and/or cellular immune response similar to those obtained using other conventional methods of administration. The invention provides for a method of inducing an immune response by an amount of vaccine which is otherwise insufficient for producing an immune response when delivered via conventional vaccine routes, e.g., intramuscular delivery.

The present disclosure also relates to methods and devices for delivering vaccines or therapeutic agents to an individual based on directly targeting the dermal space whereby such method allows improved delivery and/or improved humoral and cellular responses to the vaccines or therapeutic agents. By the use of direct intradermal (ID) administration means (hereafter referred to as dermal-access means), for example using microneedle-based injection and infusion systems, or other means to accurately target the intradermal space, the efficacy of many substances including vaccines and gene therapeutic agents can be improved when compared to traditional parental administration routes of intravenous, subcutaneous and intramuscular delivery.

Accordingly, it is one object of the invention to provide a method to accurately target the ID tissue to deliver a vaccine or a gene therapeutic agent to afford an increased immunogenic and/or therapeutic response compared to targeting the vein subcutaneous tissue or muscles. Specifically, humoral and/or cellular immune response is improved when vaccines are administered in accordance with the present invention.

It is a further object of the invention to provide a method to increased the systemic immunogenic and/or therapeutic response to vaccine or gene therapeutic agent accurately targeting the ID tissue. Specifically, humoral and/or cellular immune response is increased, compared to conventional vaccine delivery routes, e.g., intramuscular delivery.

Yet another object of the invention is to provide a method of activation of antigen presenting cells (“APC”) residing in the skin in order to effectuate an antigen-specific immune response to the vaccine by accurately targeting the ID tissue. This may, in many cases, allow for reduced doses of the substance to be administered via the ID route.

Yet another object of the present invention is to provide a method to improve the delivery of a therapeutic agent for the treatment of skin diseases, genetic skin disorders or skin cancer by accurately targeting the ID tissue. In specific embodiment, a polypeptide encoded by a genetic material is subsequently expressed in the cells within the targeted ID tissue.

Yet another object of the present invention is to provide a method to improve the delivery of a therapeutic agent for the treatment of diseases, genetic disorders, or cancers affecting tissues distant from the skin by accurately targeting the ID tissue. The resultant genetic material is subsequently expressed by the cells within the targeted ID tissue, distant therefrom or both.

Yet another object of the present invention provides a method of treating or preventing an infectious disease in a subject via ID administration of a therapeutic agent and/or a vaccine comprising a component that displays the antigenicity of an infectious agent that causes the infectious disease to induce and/or increase a humoral and/or a cellular immune response to the component in the subject.

The present invention provides a method of treating or preventing an infectious disease in a subject by delivering to the intradermal space in a subject a vaccine comprising, either or both: (i) a genetic material encoding a viral polypeptide that displays the antigenicity of the infectious agent that causes the infectious disease; and (ii) a polypeptide, or a packaged virion, that displays the antigenicity of the infectious agent that causes the infectious disease, effective to induce an immune response to the polypeptide in the subject.

In a preferred embodiment, a “prime-boost” approach is utilized to deliver the vaccines to the intradermal compartment in accordance with the methods of the invention. In particular, a priming immunization is administered comprising genetic material, e.g., plasmid DNA, encoding a viral antigen, peptide or polypeptide, followed by a secondary “boost” immunization comprising a subunit protein, a polypeptide or an inactivated virus.

These and other benefits of the invention are achieved by directly targeting delivery of the vaccines or therapeutic agents to the preferred depth for the particular therapeutic or prophylactic efficacy. The inventors have found that by specifically targeting delivery of the substance to the intradermal space, the response to vaccines and therapeutic agents can be unexpectedly improved, and can in many situations resulting in clinical advantage.

### 4. BRIEF DESCRIPTION OF THE DRAWINGS

FIG. 1 shows reporter gene activity in guinea pig skin following delivery of plasmid DNA encoding firefly luciferase. Results are shown as relative light units (RLU) per mg protein for intradermal delivery by the Mantoux method, the delivery method of the invention, and control group in which topical application of the Plasmid DNA was made to shaved skin.

FIG. 2 shows reporter gene activity in rat skin following delivery of plasmid DNA encoding firefly luciferase. Results are shown as RLU/mg protein for intradermal delivery by the microdermal delivery method (one embodiment of the invention, MDD), and control group in which an unrelated plasmid DNA was injected.

FIG. 3 shows reporter gene activity in pig skin following delivery of plasmid DNA encoding β-galactosidase. Results are shown as RLU/mg protein for intradermal delivery by the Mantoux method, by ID delivery via perpendicular insertion into skin using MDD device (34 g) or 30 g needle to depths of 1 mm and 1.5 mm, respectively, and negative control.

FIG. 4 shows total protein content at recovered skin sites in pigs following Mantoux ID and MDD delivery of reporter plasmid DNA. Control (“Negative”) is untreated skin.

FIG. 5 shows the influenza-specific serum antibody response in rats following delivery of plasmid DNA encoding influenza virus hemagglutinin in the absence of added adjuvant. Plasmid DNA was administered via ID delivery with the MDD device or via intra-muscular (IM) injection with a standard needle and syringe. “Topical” indicates control group, where the preparation was topically applied to skin.

FIG. 6 shows the influenza-specific serum antibody response in rats following delivery of plasmid DNA encoding influenza virus hemagglutinin in the presence of adjuvant. Plasmid DNA was administered via ID delivery with the MDD device or via intra-muscular (IM) injection with a standard needle and syringe. “Topical” indicates control group, where the preparation was topically applied to skin.

FIG. 7 shows the influenza-specific serum antibody response in rats following “priming” with plasmid DNA in the absence of added adjuvant followed by “boosting” with whole inactivated influenza virus in the absence of added adjuvant. Plasmid DNA or whole inactivated influenza virus was administered via ID delivery with the MDD device or via intramuscular (IM) injection with a standard needle and syringe. “Topical” indicates control group, where the preparation was topically applied to skin.

FIG. 8 shows the influenza-specific serum antibody response in rats following “priming” with plasmid DNA in the presence of added adjuvant followed by “boosting” with whole inactivated influenza virus in the absence of added adjuvant. Plasmid DNA or whole inactivated influenza virus was administered via ID delivery with the MDD device or via intra-muscular (IM) injection with a standard needle and syringe. “Topical” indicates control group, where the preparation was topically applied to skin.

FIG. 9 shows the influenza-specific serum antibody response in rats to a whole inactivated influenza virus preparation administered via ID delivery with the MDD device or via intra-muscular (IM) injection with a standard needle and syringe. “Topical” indicates control group, where the preparation was topically applied to skin.

FIG. 10 shows the influenza-specific serum antibody response in pigs to a whole inactivated influenza virus preparation administered via ID delivery with the MDD device or via intra-muscular (IM) injection with a standard needle and syringe.

FIG. 11 shows the influenza-specific serum antibody response in rats to reduced doses of a whole inactivated influenza virus preparation administered via ID delivery with the MDD device or via IM injection with a standard needle and syringe.

### 4.1 DEFINITIONS

As used herein, “intradermal” (ID) is intended to mean administration of a substance into the dermis in such a manner that the substance readily reaches the richly vascularized papillary dermis where it can be rapidly systemically absorbed, or in the case of vaccines (conventional and genetic) or gene therapeutic agents may be taken up directly by cells in the skin. In the case of genetic vaccines, intended target cells include APC (including epidermal Langerhan's cells and dermal dendritic cells). In the case of gene therapeutic agents for diseases, genetic disorders or cancers affecting tissues distant from the skin, intended target cells include keratinocytes or other skin cells capable of expressing a therapeutic protein. In the case of gene therapeutic agents for diseases, genetic disorders or cancers affecting the skin, the intended target cells include those skin cells which may be affected by the disease, genetic disorder or cancer.

As used herein, “targeted delivery” means delivery of the substance to the target depth, and includes delivery that may result in the same response in a treated individual, but result in less pain, more reproducibility, or other advantage compared to an alternate accepted means of delivery (e.g., topical, subcutaneous or intramuscular).

As used herein, an “improved response” or “increased response” include an equivalent response to a reduced amount of compound administered or an increased response to an identical amount of compound that is administered by an alternate means of delivery or any other therapeutic or immunological benefit.

The terms “needle” and “needles” as used herein are intended to encompass all such needle-like structures. The terms microcannula or microneedles, as used herein, are intended to encompass structures smaller than about 31 gauge, typically about 31-50 gauge when such structures are cylindrical in nature. Non-cylindrical structures encompassed by the term microneedles would be of comparable diameter and include pyramidal, rectangular, octagonal, wedged, and other geometrical shapes.

As used herein, the term “bolus” is intended to mean an amount that is delivered within a time period of less than ten (10) minutes. A “rapid bolus” is intended to mean an amount that is delivered in less than one minute. “Infusion” is intended to mean the delivery of a substance over a time period greater than ten (10) minutes.

The term “nucleic acids” includes polynucleotides, RNA, DNA, or RNA/DNA hybrid sequences of more than one nucleotide in either single chain or duplex form, and may be of any size that can be formulated and delivered using the methods of the present invention, Nucleic acids may be of the “antisense” type. By “nucleic acid derived entity” is meant an entity composed of nucleic acids in whole or in part.

As used herein, “vaccine” refers to vaccine or vaccine composition that may comprise one or more adjuvants. It refers to conventional or genetically engineered vaccines, including but not limited to, live vaccine, attenuated vaccine, subunit vaccine, DNA vaccine and RNA vaccine and those discussed in Section 5.2 infra.

As used herein, “therapeutic agent” or “gene therapeutic agent” include biologically active agents such as drugs, cells, medicaments comprising genetic material, genetic materials. It is an agent that is intended to be delivered into or be capable of uptake by cell(s) of the treated individual. The genetic material may be incorporated and expressed in the cells. The genetic material will ordinarily include a polynucleotide that encodes a peptide, polypeptide, protein or glycoprotein of interest, optionally contained in a vector or plasmid, operationally linked to any further nucleic acid sequences necessary for expression.

When referring to the administration of vaccines or therapeutic agents, the term “simultaneously” is generally means the administration of two dosages within the same 24 hour period, whereas “sequentially” or “subsequently” is intended to mean that the dosages are separated by more than 24 hours. It will be appreciated by those of skill in the art that simultaneous administration will generally refer to dosages administered at the same medical visit, whereas subsequently or sequentially will refer to dosages that may be separated by days, weeks, months, and occasionally years, depending on the effects of a particular vaccine or gene therapeutic. In one preferred embodiment, “sequential” or “subsequent” refers to dosages that are separated by one day to six weeks.

### 5. DETAILED DESCRIPTION

The present invention improves the clinical utility of ID delivery of vaccines and therapeutic agents to humans or animals. The methods encompass devices to directly target the intradermal space and to deliver substances to the intradermal space as a bolus or by infusion. It has been discovered that the placement of the substance within the dermis provides for efficacious and/or improved responsiveness to vaccines and therapeutic agents. The device is so designed as to prevent leakage of the substance from the skin and improve adsorption or cellular uptake within the intradermal space. The immunological response to a vaccine delivered according to the methods of the invention has been found to be equivalent to or improved over conventional IM delivery of the vaccine. These results indicate that ID administration according to the methods of the invention will in many cases provide improved clinical results, in addition to the other advantages of ID delivery.

Accordingly, the present invention provides a method of increasing a humoral and/or cellular immune response elicited by a vaccine and/or a therapeutic agent comprising administering via ID a vaccine and/or a therapeutic agent. The present invention also provides a method of producing an immune response elicited by a vaccine at a dose that is otherwise insufficient for inducing an immune response when delivered via conventional vaccine routes, e.g., intramuscular delivery.

The ability to boost or increase an immune response using the method of the present invention is desirable and advantageous. The ability to augment or amplify a subject's immune response using the methods of the present invention with a generally weak vaccine or a reduced dose of a vaccine or a gene therapeutic agent presents a safer and more feasible alternative to using a more potent vaccine or a larger dose. The methods of the invention can also aid the induction of an immune response by an amount of vaccine or therapeutic agent that is insufficient to induce an immune response if conventional delivery methods were used.

The methods of the present invention is applicable to a subject which includes a human, a primate, a horse, a cow, a sheep, a pig, a goat, a dog, a cat, a rodent, and a member of the avian species.

### 5.1 Delivery and Administration of Vaccines and Therapeutic Agents

The invention encompasses delivering a vaccine or therapeutic agent to the intradermal space of a subject's skin, which is opposite from the outer surface of the skin. In particular, for vaccines, it is preferred that delivery be at a targeted depth of just under the stratum corneum and encompassing the epidermis and upper dermis (about 0.025 mm to about 2.5 mm from the outer surface of the skin). For therapeutics that target cells in the skin, the preferred target depth depends on the particular cell being targeted; for example to target the Langerhans cells, delivery would need to encompass, at least in part, the epidermal tissue depth, which typically ranging from about 0.025 mm to about 0.2 mm from the outer surface of the skin in humans. For therapeutics and vaccines that require systemic circulation, the preferred target depth would be between, at least about 0.4 mm and most preferably at least about 0.5 mm from the outer surface of the skin up to a depth of no more than about 2.5 mm from the outer surface of the skin, more preferably, no more than about 2.0 mm and most preferably no more than about 1.7 mm from the outer surface of the skin will result delivery of the substance to the desired dermal layer. Placement of the substance predominately at greater depths and/or into the lower portion of the reticular dermis is usually considered to be less desirable.

The dermal-access means used for ID administration according to the invention is not critical as long as it provides the insertion depth into the skin of a subject necessary to provide the targeted delivery depth of the substance. In most cases, the device will penetrate the skin and to a depth of about 0.5-2 mm. The dermal-access means may comprise conventional injection needles, catheters, microcannula or microneedles of all known types, employed singularly or in multiple needle arrays. The desired therapeutic or immunogenic response is directly related to the ID targeting depth. These results can be obtained by placement of the substance in the upper region of the dermis, i.e., the papillary dermis or in the upper portion of the relatively less vascular reticular dermis such that the substance readily diffuses into the papillary dermis. Placement of a substance predominately at a depth of at least about 0.025 mm to about 2.5 mm is preferred.

By varying the targeted depth of delivery of substances by the dermal-access means, behavior of the vaccine or therapeutic agent can be tailored to the desired clinical application most appropriate for a particular patient's condition. The targeted depth of delivery of substances by the dermal-access means may be controlled manually by the practitioner, or with or without the assistance of indicator means to indicate when the desired depth is reached. Preferably however, the device has structural means for controlling skin penetration to the desired depth within the intradermal space. This is most typically accomplished by means of a widened area or hub associated with the dermal-access means that may take the form of a backing structure or platform to which the needles are attached. The length of microneedles as dermal-access means are easily varied during the fabrication process and are routinely produced. Microneedles are also very sharp and of a very small gauge, to further reduce pain and other sensation during the injection or infusion. They may be used in the invention as individual single-lumen microneedles or multiple microneedles may be assembled or fabricated in linear arrays or two-dimensional arrays as to increase the rate of delivery or the amount of substance delivered in a given period of time. Microneedles having one or more sideports are also included as dermal access means. Microneedles may be incorporated into a variety of devices such as holders and housings that may also serve to limit the depth of penetration. The dermal-access means of the invention may also incorporate reservoirs to contain the substance prior to delivery or pumps or other means for delivering the vaccine or therapeutic agent under pressure. Alternatively, the device housing the dermal-access means may be linked externally to such additional components. The dermal-access means may also include safety features, either passive or active, to prevent or reduce accidental injury.

In one embodiment of the invention, ID injection can be reproducibly accomplished using one or more narrow gauge microcannula inserted perpendicular to the skin surface. This method of delivery (“microdermal delivery” or “MDD”) is easier to accomplish than standard Mantoux-style injections and, by virtue of its limited and controlled depth of penetration into the skin, is less invasive and painful. Furthermore, similar or greater biological responses, as measured here by gene expression and immune response, can be attained using the MDD devices compared to standard needles. Optimal depth for administration of a given substance in a given species can be determined by those of skill in the art without undue experimentation.

Delivery devices that place the dermal-access means at an appropriate depth in the intradermal space, control the volume and rate of fluid delivery and provide accurate delivery of the substance to the desired location without leakage are most preferred. Micro-cannula- and microneedle-based methodology and devices are described in EP 1 092 444 A1, and U.S. Application Ser. No. 606,909, filed Jun. 29, 2000. Standard steel cannula can also be used for intra-dermal delivery using devices and methods as described in U.S. Ser. No. 417,671, filed Oct. 14, 1999, the contents of each of which are expressly incorporated herein by reference. These methods and devices include the delivery of substances through narrow gauge (about 30G) “micro-cannula” with limited depth of penetration, as defined by the total length of the cannula or the total length of the cannula that is exposed beyond a depth-limiting feature. These methods and devices provide for the delivery of substances through 30 or 31 gauge cannula, however, the present invention also employs 34G or narrower “microcannula” including if desired, limited or controlled depth of penetration means. It is within the scope of the present invention that targeted delivery of substances can be achieved either through a single microcannula or an array of microcannula (or “microneedles”), for example 3-6 microneedles mounted on an injection device that may include or be attached to a reservoir in which the substance to be administered is contained.

Using the methods of the present invention, vaccines and gene therapeutic agents may be administered as a bolus, or by infusion. It is understood that bolus administration or delivery can be carried out with rate controlling means, for example a pump, or have no specific rate controlling means, for example, user self-injection. The above-mentioned benefits are best realized by accurate direct targeted delivery of substances to the dermal tissue compartment including the epidermal tissue. This is accomplished, for example, by using microneedle systems of less than about 250 micron outer diameter, and less than 2 mm exposed length. By “exposed length” it is meant the length of the narrow hollow cannula or needle available to penetrate the skin of the patient. Such systems can be constructed using known methods for various materials including steel, silicon, ceramic, and other metals, plastic, polymers, sugars, biological and or biodegradable materials, and/or combinations thereof.

It has been found that certain features of the intradermal administration methods provide the most efficacious results. For example, it has been found that placement of the needle outlet within the skin significantly affects the clinical response to delivery of a vaccine or gene therapy agent. The outlet of a conventional or standard gauge needle with a bevel angle cut to 15 degrees or less has a relatively large “exposed height”. As used herein the term exposed height refers to the length of the opening relative to the axis of the cannula resulting from the bevel cut. When standard needles are placed at the desired depth within the intradermal space (at about 90 degrees to the skin), the large exposed height of these needle outlets causes the substance usually to effuse out of the skin due to backpressure exerted by the skin itself and to pressure built up from accumulating fluid from the injection or infusion. Typically, the exposed height of the needle outlet of the present invention is from 0 to about 1 mm. A needle outlet with an exposed height of 0 mm has no bevel cut (or a bevel angle of 90 degrees) and is at the tip of the needle. In this case, the depth of the outlet is the same as the depth of penetration of the needle. A needle outlet that is either formed by a bevel cut or by an opening through the side of the needle has a measurable exposed height. In a needle having a bevel, the exposed height of the needle outlet is determined by the diameter of the needle and the angle of the primary bevel cut (“bevel angle”). In general, bevel angles of greater than 20° are preferred, more preferably between 25° and 40°. It is understood that a single needle may have more than one opening or outlet suitable for delivery of vaccines or therapeutic agents to the dermal space.

Thus the exposed height, and for the case of a cannula with an opening through the side, its position along the axis of the cannula contributes to the depth and specificity at which a vaccine or a therapeutic agent is delivered. Additional factors taken alone or in combination with the cannula, such as delivery rate and total fluid volume delivered, contribute to the target delivery of substances and variation of such parameters to optimize results is within the scope of the present invention.

It has also been found that controlling the pressure of injection or infusion may avoid the high backpressure exerted during ID administration. By placing a constant pressure directly on the liquid interface a more constant delivery rate can be achieved, which may optimize absorption and obtain an improved response for the dosage of vaccine or therapeutic agent delivered. Delivery rate and volume can also be controlled to prevent the formation of wheals at the site of delivery and to prevent backpressure from pushing the dermal-access means out of the skin. The appropriate delivery rates and volumes to obtain these effects for a selected vaccine or therapeutic agent may be determined experimentally using only ordinary skill and without undue experimentation. Increased spacing between multiple needles allows broader fluid distribution and increased rates of delivery or larger fluid volumes.

In one embodiment, to deliver vaccine or therapeutic agent the dermal-access means is placed adjacent to the skin of a subject providing directly targeted access within the intradermal space and the vaccines or therapeutic agents are delivered or administered into the intradermal space where they can act locally or be absorbed by the bloodstream and be distributed systemically. In another embodiment, the dermal-access means is positioned substantially perpendicular to the skin surface to provide vertical insertion of one or more cannula. The dermal-access means may be connected to a reservoir containing the vaccines or therapeutic agents to be delivered. The form of the substance or substances to be delivered or administered include solutions thereof in pharmaceutically acceptable diluents or solvents, emulsions, suspensions, gels, particulates such as micro- and nanoparticles either suspended or dispersed, as well as in-situ forming vehicles of the same. Delivery from the reservoir into the intradermal space may occur either passively, without application of the external pressure or other driving means to the vaccines or therapeutic agents to be delivered, and/or actively, with the application of pressure or other driving means. Examples of preferred pressure generating means include pumps, syringes, elastomer membranes, gas pressure, piezoelectric, electromotive, electromagnetic pumping, coil springs, or Belleville springs or washers or combinations thereof. If desired, the rate of delivery of the substance may be variably controlled by the pressure-generating means. As a result, vaccine or therapeutic agent enters the intradermal space and is absorbed in an amount and at a rate sufficient to produce a clinically efficacious result.

### 5.2 Eliciting Immune Responses Via Intradermal Delivery of Vaccines or Therapeutic Agent

The present invention provides a method of increasing immune responses elicited by a vaccine and/or a therapeutic agent via delivery of vaccines or therapeutic agents to the ID space. The present invention provides a method of eliciting an immune response by administering via the ID space, a reduced dose of vaccine or therapeutic agent that is otherwise insufficient for eliciting an immune response when a conventional method via IM is used.

### 5.2.1 Immune Responses

An organism's immune system reacts with two types of responses to pathogens or other harmful agents—humoral response and cell-mediated response (See Alberts, B. et al., 1994, Molecular Biology of the Cell. 1195-96). When resting B cells are activated by antigen to proliferate and mature into antibody-secreting cells, they produce and secrete antibodies with a unique antigen-binding site. This antibody-secreting reaction is known as the humoral response. On the other hand, the diverse responses of T cells are collectively called cell-mediated immune reactions. There are two main classes of T cells—cytotoxic T cells and helper T cells. Cytotoxic T cells directly kill cells that are infected with a virus or some other intracellular microorganism. Helper T cells, by contrast, help stimulate the responses of other cells: they help activate macrophages, dendritic cells and B cells, for example (See Alberts, B. et al., 1994, Molecular Biology of the Cell. 1228). Both cytotoxic T cells and helper T cells recognize antigen in the form of peptide fragments that are generated by the degradation of foreign protein antigens inside the target cell, and both, therefore, depend on major histocompatibility complex (MHC) molecules, which bind these peptide fragments, carry them to the cell surface, and present them there to the T cells (See Alberts, B. et al., 1994, Molecular Biology of the Cell. 1228). MHC molecules are typically found in abundance on antigen-presenting cells (APCs). Antigen-presenting cells (APCs), such as macrophages and dendritic cells, are key components of innate and adaptive immune responses. Antigens are generally ‘presented’ to T cells or B cells on the surfaces of other cells, the APCs. APCs can trap lymph- and blood-borne antigens and, after internalization and degradation, present antigenic peptide fragments, bound to cell-surface molecules of the major histocompatibility complex (MHC), to T cells. APCs may then activate T cells (cell-mediated response) to clonal expansion, and these daughter cells may either develop into cytotoxic T cells or helper T cells, which in turn activate B (humoral response) cells with the same MHC-bound antigen to clonal expansion and specific antibody production (See Alberts, B. et al., 1994, Molecular Biology of the Cell. 1238-45).

Two types of antigen-processing mechanisms have been recognized. The first type involves uptake of proteins through endocytosis by APCs, antigen fragmentation within vesicles, association with class II MHC molecules and expression on the cell surface. This complex is recognized by helper T cells expressing CD4. The other is employed for proteins, such as viral antigens, that are synthesized within the cell and appears to involve protein fragmentation in the cytoplasm. Peptides produced in this manner become associated with class I MHC molecules and are recognized by cytotoxic T cells expressing CD8 (See Alberts, B. et al., 1994, Molecular Biology of the Cell. 1233-34).

Stimulation of T cells involves a number of accessory molecules expressed by both T cell and APC. Co-stimulatory molecules are those accessory molecules that promote the growth and activation of the T cell. Upon stimulation, co-stimulatory molecules induce release of cytokines, such as interleukin 1 (IL-1) or interleukin 2 (IL-2), interferon, etc., which promote T cell growth and expression of surface receptors (See Paul, 1989, Fundamental Immunology. 109-10).

Normally, APCs are quiescent and require activation for their function. The identity of signals which activate APCs is a crucial and unresolved question (See Banchereau, et al., 1998, Nature 392:245-252; Medzhitov, et al., 1998, Curr Opin Immunol. 10: 12-15).

The present inventors discovered that when influenza vaccines were delivered to the ID space, increased humoral and cellular immune responses were detected. Immunization of rats by microneedles with either DNA or conventional inactivated virus vaccines resulted in mean serum immunoglobulin (Ig) and hemagglutination inhibition antibody (HA1) titres that were 2 to 500 times greater than those obtained following IM injection.

Accordingly, one aspect of the present invention relates to a method of increasing a humoral and/or cellular immune response elicited by a vaccine and/or a therapeutic agent comprising administering to the ID space a vaccine and/or a therapeutic agent such that the humoral and/or cellular immune response is increased by 2 to 500 folds as compared to administering via IM the vaccine and/or therapeutic agent. In specific embodiments, the humoral and/or cellular immune response is increased by at least 0.5-2 times, at least 2-5 times, at least 5-10 times, at least 10-50 times, at least 50-100 times, at least 100-200 times, at least 200-300 times, at least 300-400 times or at least 400-500 times.

In specific embodiments, the invention provides methods of administering a vaccine or a therapeutic agent to the ID space to generate a mean serum immunoglobulin (Ig) and hemagglutination inhibition antibody (HAI) titers that are 2 to 500 times higher as compared to administering the vaccine or therapeutic agent via the IM route. In specific embodiments, the mean serum immunoglobulin and hemagglutination inhibition antibody (HAI) titers are increased by at least 0.5-2 times, at least 2-5 times, at least 5-10 times, at least 10-50 times, at least 50-100 times, at least 100-200 times, at least 200-300 times, at least 300-400 times or at least 400-500 times. In another specific embodiment, the invention provides methods of administering a vaccine or therapeutic agent to the ID space to generate an increased interferon-γ response (that may be 2 to 500 times higher) as compared to administering the vaccine or therapeutic agent via the IM route.

### 5.2.2 Determination of Increased Immune Response

The increase in humoral or cellular immune response induced by a vaccine that is delivered to the intradermal space according to the methods of the invention can be assessed using various methods well known in the art.

In one method, the immunogenicity of the vaccine is determined by measuring antibodies produced in response, by an antibody assay, such as an enzyme-linked immunosorbent assay (ELISA) assay. Methods for such assays are well known in the art (see, e.g., Section 2.1 of Current Protocols in Immunology, Coligan et al. (eds.), John Wiley and Sons, Inc. 1997). For example, microtitre plates (96-well Immuno Plate II, Nunc) are coated with 50 μl/well of a 0.75 μg/ml extract or lysate of a cancer cell or infected cell in PBS at 4° C. for 16 hours and at 20° C. for 1 hour. The wells are emptied and blocked with 200 μl PBS-T-BSA (PBS containing 0.05% (v/v) TWEEN 20 and 1% (w/v) bovine serum albumin) per well at 20° C. for 1 hour, then washed 3 times with PBS-T. Fifty μl/well of plasma or cerebral spinal fluid from a vaccinated animal (such as a model mouse or a human patient administered with the vaccine via the ID route or IM route is applied at 20° C. for 1 hour, and the plates are washed 3 times with PBS-T. The antigen antibody activity is then measured calorimetrically after incubating at 20° C. for 1 hour with 50 μl/well of sheep anti-mouse or anti-human immunoglobulin, as appropriate, conjugated with horseradish peroxidase diluted 1:1,500 in PBS-T-BSA and (after 3 further PBS-T washes as above) with 50 μl of an o-phenylene diamine (OPD)-H₂O₂ substrate solution. The reaction is stopped with 150 μl of 2M H₂SO₄ after 5 minutes and absorbance is determined in a photometer at 492 nm (ref. 620 nm), using standard techniques.

In another method, the “tetramer staining” assay (Altman et al., 1996, Science 274: 94-96) may be used to identify antigen-specific T-cells. For example, an MHC molecule containing a specific peptide antigen, such as a tumor-specific antigen, is multimerized to make soluble peptide tetramers and labeled, for example, by complexing to streptavidin. The MHC-peptide antigen complex is then mixed with a population of T cells obtained from a patient administered with a vaccine via the ID route or IM route. Biotin is then used to stain T cells which express the tumor-specific antigen of interest.

Furthermore, using the mixed lymphocyte target culture assay, the cytotoxicity of T cells can be tested in a 4 hour ⁵¹Cr-release assay (see Palladino et al., 1987, Cancer Res. 47:5074-5079). In this assay, the mixed lymphocyte culture is added to a target cell suspension to give different effector:target (E:T) ratios (usually 1:1 to 40:1). The target cells are pre-labeled by incubating 1×10⁶ target cells in culture medium containing 500 μCi of ⁵¹Cr per ml for one hour at 37° C. The cells are washed three times following labeling. Each assay point (E:T ratio) is performed in triplicate and the appropriate controls incorporated to measure spontaneous ⁵¹Cr release (no lymphocytes added to assay) and 100% release (cells lysed with detergent). After incubating the cell mixtures for 4 hours, the cells are pelleted by centrifugation at 200 g for 5 minutes. The amount of ⁵¹Cr released into the supernatant is measured by a gamma counter. The percent cytotoxicity is measured as cpm in the test sample minus spontaneously released cpm divided by the total detergent released cpm minus spontaneously released cpm. In order to block the MHC class I cascade a concentrated hybridoma supernatant derived from K-44 hybridoma cells (an anti-MHC class I hybridoma) is added to the test samples to a final concentration of 12.5%.

Alternatively, the ELISPOT assay can be used to measure cytokine release in vitro by cytotoxic T cells after vaccine administration. Cytokine release is detected by antibodies which are specific for a particular cytokine, such as interleukin-2, tumor necrosis factor γ or interferon-γ (for example, see Scheibenbogen et al., 1997, Int. J. Cancer, 71:932-936). The assay is carried out in a microtitre plate which has been pre-coated with an antibody specific for a cytokine of interest which captures the cytokine secreted by T cells. After incubation of T cells for 24-48 hours in the coated wells, the cytotoxic T cells are removed and replaced with a second labeled antibody that recognizes a different epitope on the cytokine. After extensive washing to remove unbound antibody, an enzyme substrate which produces a colored reaction product is added to the plate. The number of cytokine-producing cells is counted under a microscope. This method has the advantages of short assay time, and sensitivity without the need of a large number of cytotoxic T cells.

### 5.2.3. Increasing Immune Responses and Reducing Dosage of Vaccines by Delivering Vaccines to ID Space

Accordingly, the present invention relates to a method for producing an immune response in a subject by delivering to the intradermal space in a subject, a vaccine composition comprising a component against which an immune response is desired to be induced, such that an immune response to the component is produced in the subject. In specific embodiments, the immune response comprises a humoral immune response and/or a cellular immune response. In specific embodiments, the immune response is at least 0.5-2 times, at least 2-5 times, at least 5-10 times, at least 10-15 times, at least 50-100 times, at least 100-200 times, at least 200-300 times, at least 300-400 times or at least 400-500 times higher than an immune response obtained from administering the vaccine composition via the IM route. In other specific embodiments, the mean serum immunoglobulin (Ig) and hemagglutination inhibition antibody (HAI) titers are increased by at least 0.5-2 times, at least 2-5 times, at least 5-10 times, at least 10-15 times, at least 50-100 times, at least 100-200 times, at least 200-300 times, at least 300-400 times or at least 400-500 times higher than an immune response obtained from administering the vaccine composition via the IM route. In specific embodiments, the interferon-γ levels are higher than that obtained from administering the vaccine via the IM route.

The present invention further relates to a method for producing an immune response in a subject by delivering to the intradermal space in a subject, a vaccine comprising, either or both (i) a genetic material encoding a polypeptide against which an immune response is desired to be induced, e.g., a viral polypeptide; and (ii) a polypeptide, or a packaged virion, against which an immune response is desired to be induced, such that an immune response to the polypeptide is produced in the subject. In specific embodiments, the immune response comprises a humoral immune response and/or a cellular immune response. In specific embodiments, the immune response is at least 0.5-2 times, at least 2-5 times, at least 5-10 times, at least 10-15 times, at least 50-100 times, at least 100-200 times, at least 200-300 times, at least 300-400 times or at least 400-500 times higher than an immune response obtained from administering the vaccine composition via the IM route. In other specific embodiments, the mean serum immunoglobulin (Ig) and hemagglutination inhibition antibody (HAI) titers are increased by at least 0.5-2 times, at least 2-5 times, at least 5-10 times, at least 10-15 times, at least 50-100 times, at least 100-200 times, at least 200-300 times, at least 300-400 times or at least 400-500 times higher than an immune response obtained from administering the vaccine composition via the IM route. In specific embodiments, the interferon-γ levels are higher than that obtained from administering the vaccine composition via the IM route.

Still further, the present invention relates to a method for producing an immune response in a subject by delivering to the intradermal space in a subject, a vaccine comprising, either or both (i) a genetic material encoding a polypeptide against which an immune response is desired to be induced e.g., a viral polypeptide; and (ii) a polypeptide, or a packaged virion, against which an immune response is desired to be induced, such that an immune response to the polypeptide is produced in the subject. In specific embodiments, the dose of the genetic material administered to the ID space is less than 0.5-1 μg, less than 1-2 μg, less than 2-4 μg, less than 4-10 μg, less than 10-20 μg, less than 20-40 μg, less than 40-60 μg, or less than 60-80 μg. In specific embodiments, the dose of the polypeptide or a packaged virion administered to the ID space is less than 0.005-0.0 μg, less than 0.01-0.05 μg, less than 0.05-0.1 μg, less than 0.1-0.5 μg, less than 0.5-0.8 μg, less than 1-2 μg, less than 1-2 μg, less than 2-4 μg, less than 4-10 μg, less than 10-20 μg, less than 20-40 μg, less than 40-60 μg, or less than 60-80 μg.

The present invention enables administration of a reduced dose of vaccine to elicit an immune response in a subject. This is beneficial especially for reduced cost of vaccination, increased availability of vaccines to more subjects, especially for vaccines that are expensive or difficult to produce. In specific embodiments, the invention provides methods of eliciting an immune response by an initial immunization (prime) by boost in immunization with administering a DNA vaccine at doses as low as 1 g followed by an inactivated virus at doses as low as 0.01 μg. This dose is 100 less than that required to generate similar immune responses when the DNA vaccine and inactivated virus are administered via the IM route.

In a specific embodiment, the invention provides a method to elicit an immune response by administering an initial immunization (prime) using a DNA vaccine at doses that are less than 0.5-1 μg, less than 1-2 μg, less than 2-4 μg, less than 4-10 μg, less than 10-20 μg, less than 20-40 μg, less than 40-60 μg, or less than 60-80 μg, and then followed by a boost immunization with an inactivated virus at doses that are less than 0.005-0.01 μg, less than 0.01-0.05 μg, less than 0.05-0.1 μg, less than 0.1-0.5 μg, or less than 0.5-0.8 μg.

In specific embodiments, the prime immunization and the boost immunization according to the method of the present invention generate an humoral and/or cellular immune response that is increased by at least 2-5 times, at least 5-10 times, at least 10-15 times, at least 50-100 times, at least 100-200 times, at least 200-300 times, at lest 300-400 times, or at least 400-500 times as compared to immunizations using the IM route. In specific embodiments, the invention provides methods of administering a vaccine to the ID space to generate a mean serum immunoglobulin (Ig) and hemagglutination inhibition antibody (HAI) titers that are increased by at least 2-5 times, at least 5-10 times, at least 10-50 times, at least 50-100 times, at least 100-200 times, at least 200-300 times, at least 300-400 times or at least 400-500 times compared to administration of vaccines via the IM route. In another specific embodiment, prime and boost immunizations generate an increased level of INF-γ, indicating an increased cell-mediated immune response.

### 5.3 Vaccines and Therapeutic Agents

Substances that may be delivered according to the methods of the invention include vaccines, with or without carriers, adjuvants and vehicles. Vaccines or immunogenic preparations useful for the methods of the present invention encompass single or multivalent vaccines, including bivalent and trivalent vaccines. Therapeutic agents may include prophylactic and therapeutic antigens including but not limited to subunit proteins, peptides and polysaccharides, polysaccharide conjugates, toxoids, genetic based vaccines, live attenuated bacteria or viruses, mutated bacteria or viruses, reassortant bacteria or viruses, inactivated bacteria or viruses, whole cells or components thereof (e.g., mammalian cells), cellular vaccines (e.g., autologous dendritic cells), or components thereof (for example, exosomes, dexosomes, membrane fragments, or vesicles), live viruses, live bacteria, anthrax, arthritis, cholera, diphtheria, dengue, tetanus, lupus, multiple sclerosis, parasitic diseases, psoriasis, Lyme disease, meningococcus, measles, mumps, rubella, varicella, yellow fever, respiratory syncytial virus, tick borne Japanese encephalitis, pneumococcus, smallpox, streptococcus, staphylococcus, typhoid, influenza, hepatitis, including hepatitis A, B, C and E, otitis media, rabies, polio, HIV, parainfluenza, rotavirus, Epstein Barr Virus, CMV, chlamydia, non-typeable haemophilus, haemophilus influenza B (HIB), moraxella catarrhalis, human papilloma virus, tuberculosis including BCG, gonorrhoeae, asthma, atherosclerosis, malaria, E. coli, Alzheimer's Disease, H. Pylori, salmonella, diabetes, cancer, herpes simplex, human papilloma, Yersinia pestis, traveler's diseases, West Nile encephalitis, Camplobacter, C. difficile, Kunjin virus, Powassan virus, Kyasanur Forest Disease virus, and Omsk Hemorrhagic Fever Virus, and parasite antigens (e.g., malaria).

More preferred are vaccines or immunogenic formulations that provide protection against respiratory tract diseases, such as but not limited to, respiratory syncytial virus vaccines, influenza vaccines, measles vaccines, mumps vaccines, rubella vaccines, pneumococcal vaccines, rickettsia vaccines, staphylococcus vaccines, whooping cough vaccines, severe acute respiratory symptom (“SARS”) vaccines, or vaccines against respiratory tract cancers.

In other preferred embodiments, the vaccines or immunogenic formulations are pediatric vaccines. In more preferred embodiments, the pediatric vaccines are administered using the methods of the present invention at the recommended ages. For example, at two, four or six months of age, the vaccines are DtaP, Hib, Polio and Hepatitis B. At twelve or fifteen months of age, the vaccines are Hib, Polio, MMRII®, Varivax®, and Hepatitis B. Vaccines that may be used in the methods of the present invention are reviewed in various publications, e.g. The Jordan Report 2000, Division of Microbiology and Infectious Diseases, National Institute of Alergy and Infectious Diseases, National Institutes of Health.

The vaccines used in the methods of the invention may comprise one or more antigenic or immunogenic agent, against which an immune response is desired. Vaccine formulations that are useful for the methods of the present invention comprise recombinant viruses encoded by viral vectors derived from the genome of a virus, such as adenovirus, retrovirus, alphavirus, flavivirus, and vaccina virus. A recombinant virus may be encoded by endogenous or native genomic sequences and/or non-native genomic sequences of a virus. A native or genomic sequence is one that is different from the native or endogenous genomic sequence due to one or more mutations, including, but not limited to, point mutations, rearrangements, insertions, deletions etc., to the genomic sequence that may or may not result in a phenotypic change. A recombinant virus may be encoded by a nucleotide sequence in which heterologous nucleotide sequences have been added to the genome or in which endogenous or native nucleotide sequences have been replaced with heterologous nucleotide sequences.

Preferably, epitopes that induce a protective immune response to any of a variety of pathogens, or antigens that bind neutralizing antibodies may be used in the methods of the present invention. For example, heterologous gene sequences of influenza and parainfluenza hemagglutinin neuramimidase and fusion glycoproteins such as the HN and F genes of human PIV3 may be used in the methods of the present invention.

The therapeutic agents that are useful in the methods of the present invention may comprise antigens or nucleic acid molecules comprising nucleic acid sequences that encode tumor antigens. These therapeutic agents may be used to generate an immune response against tumor cells. Other therapeutic agents that may be useful express tumor-associated antigens (TAAs), including but not limited to, human tumor antigens recognized by T cells (Robbins and Kawakami, 1996, Curr. Opin. Immunol. 8:628-636, incorporated herein by reference in its entirety), melanocyte lineage proteins, including gp100, MART-1/MelanA, TRP-1 (gp75), tyrosinase; Tumor-specific widely shared antigens, MAGE-1, MAGE-3, BAGE, GAGE-1, GAGE-1, N-acetylglucosaminyltransferase-V, p15; Tumor-specific mutated antigens, β-catenin, MUM-1, CDK4; Nonmelanoma antigens for breast, ovarian, cervical and pancreatic carcinoma, HER-2/neu, human papillomavirus-E6, -E7, MUC-1. In specific embodiments, the methods of the present invention use vaccines that are specific to or genetic materials that encode a cancer antigen, such as KS 1/4 pan-carcinoma antigen (Perez and Walker, 1990, J. Immunol. 142:3662-3667; Bumal, 1988, Hybridoma 7(4):407-415); ovarian carcinoma antigen (CA125) (Yu et al., 1991, Cancer Res. 51(2):468-475); prostatic acid phosphate (Tailor et al., 1990, Nucl. Acids Res. 18(16):4928); prostate specific antigen (Henttu and Vihko, 1989, Biochem. Biophys. Res. Comm. 160(2):903-910; Israeli et al., 1993, Cancer Res. 53:227-230); melanoma-associated antigen p97 (Estin et al., 1989, J. Natl. Cancer Instit. 81(6):445-446); melanoma antigen gp75 (Vijayasardahl et al., 1990, J. Exp. Med. 171(4):1375-1380); high molecular weight melanoma antigen (HMW-MAA) (Natali et al., 1987, Cancer 59:55-63; Mittelman et al., 1990, J. Clin. Invest. 86:2136-2144); prostate specific membrane antigen; carcinoembryonic antigen (CEA) (Foon et al., 1994, Proc. Am. Soc. Clin. Oncol. 13:294); polymorphic epithelial mucin antigen; human milk fat globule antigen; a colorectal tumor-associated antigen, such as CEA, TAG-72 (Yokata et al., 1992, Cancer Res. 52:3402-3408), CO 17-1A (Ragnhammar et al., 1993, Int. J. Cancer 53:751-758); GICA 19-9 (Herlyn et al., 1982, J. Clin. Immunol. 2:135), CTA-1 and LEA; Burkitt's lymphoma antigen-38.13; CD19 (Ghetie et al., 1994, Blood 83:1329-1336); human B-lymphoma antigen-CD20 (Reff et al., 1994, Blood 83:435-445); CD33 (Sgouros et al., 1993, J. Nucl. Med. 34:422-430); melanoma specific antigens such as ganglioside GD2 (Saleh et al., 1993, J. Immunol., 151, 3390-3398), ganglioside GD3 (Shitara et al., 1993, Cancer Immunol. Immunother. 36:373-380), ganglioside GM2 (Livingston et al., 1994, J. Clin. Oncol. 12:1036-1044), ganglioside GM3 (Hoon et al., 1993, Cancer Res. 53:5244-5250); tumor-specific transplantation type of cell-surface antigen (TSTA) such as virally-induced tumor antigens including T-antigen DNA tumor viruses and envelope antigens of RNA tumor viruses; oncofetal antigen-alpha-fetoprotein such as CEA of colon, bladder tumor oncofetal antigen (Hellstrom et al., 1985, Cancer. Res. 45:2210-2188); differentiation antigen such as human lung carcinoma antigen L6, L20 (Hellstrom et al., 1986, Cancer Res. 46:3917-3923); antigens of fibrosarcoma, human leukemia T cell antigen-Gp37 (Bhattacharya-Chatterjee et al., 1988, J. of Immunospecifically. 141:1398-1403); neoglycoprotein, sphingolipids, breast cancer antigen such as EGFR (Epidermal growth factor receptor), HER2 antigen (p185^{HER2}), polymorphic epithelial mucin (PEM) (Hilkens et al., 1992, Trends in Bio. Chem. Sci. 17:359); malignant human lymphocyte antigen-APO-1 (Bernhard et al., 1989, Science 245:301-304); differentiation antigen (Feizi, 1985, Nature 314:53-57) such as I antigen found in fetal erythrocytes, primary endoderm, I antigen found in adult erythrocytes and preimplantation embryos, I(Ma) found in gastric adenocarcinomas, M18, M39 found in breast epithelium, SSEA-1 found in myeloid cells, VEP8, VEP9, Myl, VIM-D5, D₁56-22 found in colorectal cancer, TRA-1-85 (blood group H), C14 found in colonic adenocarcinoma, F3 found in lung adenocarcinoma, AH6 found in gastric cancer, Y hapten, Le^{y} found in embryonal carcinoma cells, TL5 (blood group A), EGF receptor found in A431 cells, E₁ series (blood group B) found in pancreatic cancer, FC10.2 found in embryonal carcinoma cells, gastric adenocarcinoma antigen, CO-514 (blood group Le^{a}) found in Adenocarcinoma, NS-10 found in adenocarcinomas, CO-43 (blood group Le^{b}), G49 found in EGF receptor of A431 cells, MH2 (blood group ALe^{b}/Le^{y}) found in colonic adenocarcinoma, 19.9 found in colon cancer, gastric cancer mucins, T₅A₇ found in myeloid cells, R₂₄ found in melanoma, 4.2, GD3, D1.1, OFA-1, G_{M2}, OFA-2, GD2, and M1:22:25:8 found in embryonal carcinoma cells, and SSEA-3 and SSEA-4 found in 4 to 8-cell stage embryos. In one embodiment, the antigen is a T-cell receptor-derived peptide from a cutaneous T-cell lymphoma (see, Edelson, 1998, The Cancer Journal 4:62).

In another specific embodiment, the methods of the present invention use vaccines that are specific to or genetic materials that encode an infectious disease agent, such as: influenza virus hemagglutinin (Genbank accession no. J02132; Air, 1981, Proc. Natl. Acad. Sci. USA 78:7639-7643; Newton et al., 1983, Virology 128:495-501); human respiratory syncytial virus G glycoprotein (Genbank accession no. Z33429; Garcia et al., 1994, J. Virol.; Collins et al., 1984, Proc. Natl. Acad. Sci. USA 81:7683); core protein, matrix protein or other protein of Dengue virus (Genbank accession no. M19197; Hahn et al., 1988, Virology 162:167-180); measles virus hemagglutinin (Genbank accession no. M81899; Rota et al., 1992, Virology 188:135-142); herpes simplex virus type 2 glycoprotein gB (Genbank accession no. M14923; Bzik et al., 1986, Virology 155:322-333); poliovirus I VP1 (Emini et al., 1983, Nature 304:699); an envelope glycoprotein of HIV I (Putney et al., 1986, Science 234:1392-1395); hepatitis B surface antigen (Itoh et al., 1986, Nature 308:19; Neurath et al., 1986, Vaccine 4:34); diptheria toxin (Audibert et al., 1981, Nature 289:543); streptococcus 24M epitope (Beachey, 1985, Adv. Exp. Med. Biol. 185:193); gonococcal pilin (Rothbard and Schoolnik, 1985, Adv. Exp. Med. Biol. 185:247); pseudorabies virus g50 (gpD); pseudorabies virus II (gpB); pseudorabies virus gill (gpC); pseudorabies virus glycoprotein H; pseudorabies virus glycoprotein E; transmissible gastroenteritis glycoprotein 195; transmissible gastroenteritis matrix protein; swine rotavirus glycoprotein 38; swine parvovirus capsid protein; Serpulina hydodysenteriae protective antigen; bovine viral diarrhea glycoprotein 55; Newcastle disease virus hemagglutinin-neuramimidase; swine flu hemagglutinin; swine flu neuramimidase; foot and mouth disease virus; hog colera virus; swine influenza virus; African swine fever virus; Mycoplasma hyopneumoniae; infectious bovine rhinotracheitis virus (e.g., infectious bovine rhinotracheitis virus glycoprotein E or glycoprotein G), or infectious laryngotracheitis virus (e.g., infectious laryngotracheitis virus glycoprotein G or glycoprotein I); a glycoprotein of La Crosse virus (Gonzales-Scarano et al., 1982, Virology 120:42); neonatal calf diarrhea virus (Matsuno and Inouye, 1983, Infection and Immunity 39:155); Venezuelan equine encephalomyelitis virus (Mathews and Roehrig, 1982, J. Immunol. 129:2763); punta toro virus (Dalrymple et al., 1981, in Replication of Negative Strand Viruses, Bishop and Compans (eds.), Elsevier, NY, p. 167); murine leukemia virus (Steeves et al., 1974, J. Virol. 14:187); mouse mammary tumor virus (Massey and Schochetman, 1981, Virology 115:20); hepatitis B virus core protein and/or hepatitis B virus surface antigen or a fragment or derivative thereof (see, e.g., U.K. Patent Publication No. GB 2034323A published Jun. 4, 1980; Ganem and Varmus, 1987, Ann. Rev. Biochem. 56:651-693; Tiollais et al., 1985, Nature 317:489-495); antigen of equine influenza virus or equine herpesvirus (e.g., equine influenza virus type A/Alaska 91 neuramimidase, equine influenza virus type A/Miami 63 neuramimidase; equine influenza virus type A/Kentucky 81 neuramimidase; equine herpesvirus type 1 glycoprotein B; equine herpesvirus type 1 glycoprotein D); antigen of bovine respiratory syncytial virus or bovine parainfluenza virus (e.g., bovine respiratory syncytial virus attachment protein (BRSV G); bovine respiratory syncytial virus fusion protein (BRSV F); bovine respiratory syncytial virus nucleocapsid protein (BRSV N); bovine parainfluenza virus type 3 fusion protein; and the bovine parainfluenza virus type 3 hemagglutinin neuramimidase); bovine viral diarrhea virus glycoprotein 48 or glycoprotein 53.

The present invention relates to a method for delivering therapeutic agents to the intradermal space in a subject such that adsorption or cellular uptake of therapeutic agents is improved as compared to delivery via IM, IV, or SC. Therapeutic agents that are useful for the methods of the present invention includes antibiotic, antifungal, anti-viral or other drug useful in treating the particular disease.

### 5.3.1 Vaccine Formulations

Vaccine formulations that are useful in the methods of the present invention are suitable for administration to elicit a protective immune (humoral and/or cell mediated) response against certain antigens, as described in section 5.3 supra.

Suitable preparations of such vaccines include injectables, either as liquid solutions or suspensions; solid forms suitable for solution in, or suspension in, liquid prior to injection, may also be prepared. The preparation may also be emulsified, or the polypeptides encapsulated in liposomes. The active immunogenic ingredients are often mixed with excipients which are pharmaceutically acceptable and compatible with the active ingredient. Suitable excipients are, for example, water, saline, buffered saline, dextrose, glycerol, ethanol, sterile isotonic aqueous buffer or the like and combinations thereof. In addition, if desired, the vaccine preparation may also include minor amounts of auxiliary substances such as wetting or emulsifying agents, pH buffering agents, and/or adjuvants which enhance the effectiveness of the vaccine.

Examples of adjuvants which may be effective, include, but are not limited to: aluminim hydroxide, N-acetyl-muramyl-L-threonyl-D-isoglutamine (thr-MDP), N-acetyl-nor-muramyl-L-alanyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine.

### 5.5 Treatment of Infectious Disease

The present invention provides a method of treating or preventing an infectious disease in a subject by delivering a therapeutic agent to the intradermal space in a subject such that the therapeutic agent, i.e., the vaccine, is more effective as compared to conventional delivery routes, e.g., IM, IV or SC.

The invention also provides methods of treating or preventing an infectious disease by administering to a subject via the ID space a vaccine comprising a component that displays the antigenicity of an infectious disease agent that causes the infectious disease (e.g., an immunogenic amount of an antigen on the infection agent) to induce an immune response to the component in the subject.

The present invention provides a method of treating or preventing an infectious disease in a subject by delivering to the intradermal space in a subject, a vaccine comprising, either or both: (i) a genetic material encoding a viral polypeptide that displays the antigenicity of the infectious agent that causes the infectious disease; and (ii) a polypeptide, or a packaged virion, that displays the antigenicity of the infectious agent that causes the infectious disease, effective to induce an immune response to the polypeptide in the subject.

In a preferred embodiment, a “prime-boost” approach is utilized to deliver the vaccines to the intradermal compartment in accordance with the methods of the invention. In particular, a priming immunization is administered comprising genetic material, e.g., plasmid DNA, encoding a viral antigen, peptide or polypeptide, followed by a secondary “boost” immunization comprising a subunit protein, a polypeptide or an inactivated virus.

In preferred embodiments, infectious agents include, but are not limited to, viruses, bacteria, fungi, protozoa, and parasites. the pathogen which binds to the cellular receptor. Pathogens that causes infectious diseases include B-lymphotropic papovavirus (LPV), Bordatella pertussis, Boma Disease virus (BDV), Bovine coronavirus, Choriomeningitis virus, Dengue virus, E. coli, Ebola, Echovirus 1, Echovirus-11 (EV), Endotoxin (LPS), Enteric bacteria, Enteric Orphan virus, Enteroviruses, Feline leukemia virus, Foot and mouth disease virus, Gibbon ape leukemia virus (GALV), Gram-negative bacteria, Heliobacter pylori, Hepatitis B virus (HBV), Herpes Simplex Virus, HIV-1, Human cytomegalovirus, Human coronovirus, Influenza A, B & C, Legionella, Leishmania mexicana, Listeria monocytogenes, Measles virus, Meningococcus, Morbilliviruses, Mouse hepatitis virus, Murine leukemia virus, Murine gamma herpes virus, Murine retrovirus, Murine coronavirus mouse hepatitis virus, Mycobacterium avium-M, Neisseria gonorrhoeae, Newcastle disease virus, Parvovirus B 19, Plasmodium falciparum, Pox Virus, Pseudomonas, Rotavirus, Samonella typhiurium, Shigella, Streptococci, T-helper cells type 1, T-cell lymphotropic virus 1, and Vaccinia virus.

In preferred embodiments, viral diseases that can be treated using the methods of the present invention include, but are not limited to, those caused by hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, hantavirus, coxsachie virus, mumps virus, measles virus, rubella virus, polio virus, human immunodeficiency virus type I (HIV-I), and human immunodeficiency virus type II (HIV-II), any picornaviridae, enteroviruses, caliciviridae, any of the Norwalk group of viruses, togaviruses, such as Dengue virus, alphaviruses, flaviviruses, coronaviruses, rabies virus, Marburg viruses, ebola viruses, parainfluenza virus, orthomyxoviruses, bunyaviruses, arenaviruses, reoviruses, rotaviruses, orbiviruses, human T cell leukemia virus type I, human T cell leukemia virus type II, simian immunodeficiency virus, lentiviruses, polyomaviruses, parvoviruses, Epstein-Barr virus, human herpesvirus-6, cercopithecine herpes virus 1 (B virus), poxviruses, and encephalitis.

In preferred embodiments, bacterial diseases that can be treated using the methods of the present invention include those caused by, but not limited to, gram negative or gram positive bacteria, mycobacteria rickettsia, mycoplasma, Shigella spp., Neisseria spp. (e.g., Neisseria mennigitidis and Neisseria gonorrhoeae), legionella, Vibrio cholerae, Streptococci, such as Streptococcus pneumoniae, corynebacteria diphtheriae, clostridium tetani, bordetella pertussis, Haemophilus spp. (e.g., influenzae), Chlamydia spp., Enterotoxigenic Escherichia coli, etc. and bacterial diseases Syphillis, Lyme's disease.

In preferred embodiments, protozoal diseases that can be treated using the methods of the present invention include those cause by, but not limited to, plasmodia, Eimeria, Leishmannia, kokzidioa, and trypanosoma, and fungi such as Candida.

### 5.6. Kits

Typically, to administer vaccine or other medicament a practitioner will remove the appropriate volume from a vial sealed with a septa using a syringe. This same syringe is then used administer the vaccine to the patient. However, a microneedle or microcannula, typically between 0.1 and 2 mm in length, in addition to being somewhat unsuitable in length to completely penetrate the septa, is generally too fragile to puncture a septum of a vial to extract medicament while maintaining sufficient sharpness and straightness to subsequently be used on a patient. Use of such microdevices in puncturing septa also may result in clogging of the bore of the needle. In addition, the narrow gauge, typically 31 to 50 gauge, of the microcannula greatly reduces the volumetric capacity that can traverse the needle into the syringe, for example. This would be inconvenient to most practitioners who are accustomed to rapid transfer of liquids from vials using conventional devices and thus would greatly increase the amount of time the practitioner would spend with the patient. Additional factors to be considered in the widespread use of microdevices include the necessity to reformulate most drugs and vaccines to accommodate the reduced total volume (10-100 μl) used or delivered by microdevices. Thus it would be desirable to provide for a kit including the device either in combination with or adapted to integrate therewith, the substance to be delivered.

Kits and the like comprising the instrument of administration and the therapeutic composition are well known in the art. However, the application of minimally invasive, ID microdevices for the delivery of vaccines and therapeutic agents clearly present an immediate need for coupling the device with the formulation to provide safe, efficacious, and consistent means for administering formulations for enabling immunogenic and therapeutic responses.

The kit provided by the invention comprises a delivery device having at least one hollow microneedle designed to intradermally deliver a substance to a depth between 0.025 and 2 mm which is adapted so that the microneedle is or can be placed in fluid connection with a reservoir adapted for containing a dosage of a vaccine or therapeutic agent. In a preferred embodiment, the kit also contains an effective dosage of a vaccine or therapeutic agent, optionally contained in a reservoir that is an integral part of, or is capable of being functionally attached to, the delivery device. The hollow microneedle is preferably between about 31 to 50 gauge, and may be part of an array of, for example, 3-6 microneedles.

In a particularly preferred embodiment, the kit of the invention comprises a hub portion being attachable to the prefillable reservoir storing the vaccine; at least one microneedle supported by said hub portion and having a forward tip extending away from said hub portion; and a limiter portion surrounding said microneedle(s) and extending away from said hub portion toward said forward tip of said microneedle(s), said limiter including a generally flat skin engaging surface extending in a plane generally perpendicular to an axis of said microneedle(s) and adapted to be received against the skin of a mammal to administer an intradermal injection of the vaccine, said microneedle(s) forward tip(s) extending beyond said skin engaging surface a distance approximately 0.5 mm to 2.0 mm wherein said limiter portion limits penetration of the microneedle(s) into the dermal layer of skin of the mammal.

To use a kit as envisioned by the instant invention the practitioner would break a hermetic seal to provide access to the microdevice and optionally, the vaccine or therapeutic agent. The composition may be preloaded within the microdevice in any form including but not limited to gel, paste, oil, emulsion, particle, nanoparticle, microparticle, suspension or liquid. The composition may be separately packaged within the kit package, for example, in a reservoir, vial, tube, blister, pouch or the like. One or more of the constituents of the formulation may be lyophilized, freeze-dried, spray freeze-dried, or in any other reconstitutable form. Various reconstitution media, cleansing or disinfective agents, or topical steriliants (alcohol wipes, iodine) can further be provided if desired. The practitioner would then load or integrate the substance if necessary into the device and then administer the formulation to the patient using the ID injection microdevice.

In a specific embodiment, the invention comprises kits comprising a device for intradermal delivery and vaccine formulation. In another specific embodiment, the invention provides a kit for use in inducing an immune response to a viral antigen in a subject, said kit comprising: (a) a protein expressed by an influenza virus and (b) a device that targets the intradermal compartment of the subject's skin.

### 6. EXAMPLES

Having described the invention in general, the following specific but not limiting examples and reference to the accompanying Figures set forth various examples for practicing the invention.

A representative example of dermal-access microdevice (MDD device) comprising a single needle were prepared from 34 gauge steel stock (MicroGroup, Inc., Medway, Mass.) and a single 28° bevel was ground using an 800 grit carborundum grinding wheel. Needles were cleaned by sequential sonication in acetone and distilled water, and flow-checked with distilled water. Microneedles were secured into small gauge catheter tubing (Maersk Medical) using UV-cured epoxy resin. Needle length was set using a mechanical indexing plate, with the hub of the catheter tubing acting as a depth-limiting control and was confirmed by optical microscopy. The exposed needle length was adjusted to 1 mm using an indexing plate. Connection to the syringe was via an integral Luer adapter at the catheter inlet. During injection, needles were inserted perpendicular to the skin surface, and were held in place by gentle hand pressure for bolus delivery. Devices were checked for function and fluid flow both immediately prior to and post injection. A 30/31 gauge intradermal needle device with 1.5 mm exposed length controlled by a depth limiting hub as described in EP 1 092 444 A1 was also used in some Examples.

### Example 1

### ID Delivery and Expression of Model Genetic Therapeutic/Prophylactic Agents, Guinea Pig Model

Uptake and expression of DNA by cells in vivo are critical to effective gene therapy and genetic immunization. Plasmid DNA encoding the reporter gene, firefly luciferase, was used as a model gene therapeutic agent (Aldevron, Fargo, N. Dak.). DNA was administered to Hartley guinea pigs (Charles River, Raleigh, N.C.) intradermally (ID) via the Mantoux (ID-Mantoux) technique using a standard 30G needle or was delivered ID via MDD (ID-MDD) using a 34G steel micro-cannula of 1 mm length (MDD device) inserted approximately perpendicular. Plasmid DNA was applied topically to shaved skin as a negative control (the size of the plasmid is too large to allow for passive uptake into the skin). Total dose was 100 μg per animal in total volume of 40 μl PBS delivered as a rapid bolus injection (<1 min) using a icc syringe. Full thickness skin biopsies of the administration sites were collected 24 hr. following delivery, were homogenized and further processed for luciferase activity using a commercial assay (Promega, Madison, Wis.). Luciferase activity was normalized for total protein content in the tissue specimens as determined by BCA assay (Pierce, Rockford, Ill.) and is expressed as Relative Light Units (RLU) per mg of total protein (n=3 animals per group for Mantoux and Negative control and n=6 for MDD device).

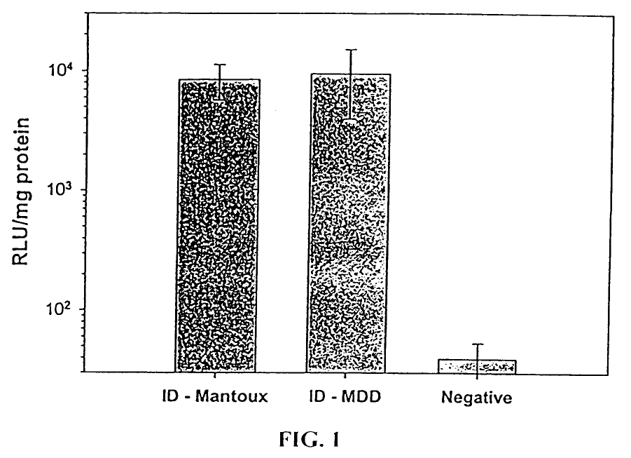

The results (FIG. 1) demonstrate strong luciferase expression in both ID injection groups. Mean luciferase activity in the MDD and Mantoux groups were 240- and 220-times above negative controls, respectively. Luciferase expression levels in topical controls were not significantly greater than in untreated skin sites (data not shown). These results demonstrate that the method of the present invention using MDD devices is at least as effective as the Mantoux technique in delivering genetic materials to the ID tissue and results in significant levels of localized gene expression by skin cells in vivo.

### Example 2

### ID Delivery and Expression of Model Genetic Therapeutic/Prophylactic Agents, Rat Model

Experiments similar (without Mantoux control) to those described in Example 1 above were performed in Brown-Norway rats (Charles River, Raleigh, N.C.) to evaluate the utility of this platform across multiple species. The same protocol was used as in Example 1, except that the total plasmid DNA load was reduced to 50 μg in 50 μl volume of PBS. In addition, an unrelated plasmid DNA (encoding b-galactosidase) injected into the ID space (using the MDD device) was used as negative control. (n=4 animals per group). Luciferase activity in skin was determined as described in Example 1 above.

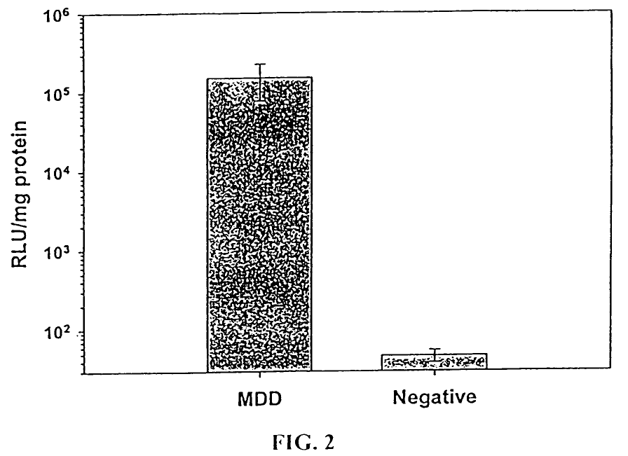

The results, shown in FIG. 2, demonstrate very significant gene expression following ID delivery via the MDD device. Luciferase activity in recovered skin sites was >3000-fold greater than in negative controls. These results further demonstrate the utility of the method of the present invention in delivering gene based entities in vivo, resulting in high levels of gene expression by skin cells.

### Example 3

### ID Delivery and Expression of Model Genetic Therapeutic/Prophylactic Agents, Pig Model

The pig has long been recognized as a preferred animal model for skin based delivery studies. Swine skin is more similar to human skin in total thickness and hair follicle density than is rodent skin. Thus, the pig model (Yorkshire swine; Archer Farms, Belcamp, Md.) was used as a means to predict the utility of this system in humans. Experiments were performed as above in Examples 1 and 2, except using a different reporter gene system, β-galactosidase (Aldevron, Fargo, N. Dak.). Total delivery dose was 50 μg in 50 μl volume. DNA was injected using the following methods: (i) via Mantoux method using a 30G needle and syringe; (ii) by ID delivery via perpendicular insertion into skin using a 30/31G needle equipped with a feature to limit the needle penetration depth to 1.5 mm; and (iii) by ID delivery via perpendicular insertion into skin using a 34G needle equipped with a feature to limit the needle penetration depth to 1.0 mm (MDD device). The negative control group consisted of ID delivery by (i)-(iii) of an unrelated plasmid DNA encoding firefly luciferase. (n=11 skin sites from 4 pigs for the ID Mantoux group; n=11 skin sites from 4 pigs for ID, 30/31G, 1.5 mm device; n=10 skin sites from 4 pigs for ID, 34G, 1 mm device; n=19 skin sites from 4 pigs for negative control.) For the negative control, data from all 3 ID delivery methods were combined since all 3 methods generated comparable results.

Reporter gene activity in tissue was determined essentially as described in Example 1, except substituting the β-galactosidase detection assay (Applied Biosystems, Foster City, Calif.) in place of the luciferase assay.

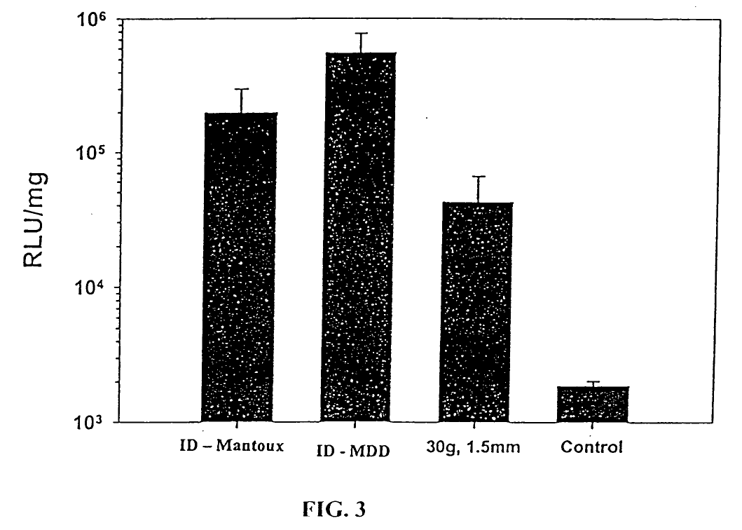

The results, shown in FIG. 3, indicate strong reporter gene expression in skin following all 3 types of ID delivery. Responses in the ID-Mantoux group were 100-fold above background, compared to a 300-fold increase above background in the ID, 34G, 1 mm (MDD) group and 20-fold increase above background in the ID, 30G, 1.5 mm (30 g, 1.5 mm) group. Total reporter gene expression by skin cells, as measured by reporter gene mean activity recovered from excised skin tissue biopsies, was strongest in the ID, 34G, 1 mm (MDD) group at 563,523 RLU/mg compared to 200,788 RLU/mg in the ID, 30G Mantoux group, 42,470 RLU/mg in the ID (30G, 1.5 mm) group and 1,869 RLU/mg in the negative controls. Thus, ID delivery via perpendicular insertion of a 34G, 1.0 mm needle (MDD) results in superior uptake and expression of DNA by skin cells as compared to the standard Mantoux style injection or a similar perpendicular needle insertion and delivery using a longer (1.5 mm), wider diameter (30G) needle. Similar studies using these 3 devices and methods to deliver visible dyes also demonstrate that the 34G, 11.0 mm needle results in more consistent delivery to the ID tissue than the other 2 needles/methods and results in less “spill-over” of the administered dose into the subcutaneous (SC) tissue.

These differences were unexpected since all 3 devices and methods theoretically target the same tissue space. However, it is much more difficult to control the depth of delivery using a lateral insertion (Mantoux) technique as compared to a substantially perpendicular insertion technique that is achieved by controlling the length of the cannula via the depth-limiting hub. Further, the depth of needle insertion and exposed height of the needle outlet are important features associated with reproducible ID delivery without SC “spill-over” or leakage on the skin surface.

These results further demonstrate the utility of the methods of the present invention in delivering gene based entities in larger mammals in vivo, resulting in high levels of gene expression by skin cells. In addition, the similarities in skin composition between pigs and humans indicate that comparable clinical improvements should be obtained in humans.

### Example 4

### Indirect Measurement of Localized Tissue Damage Following ID Delivery

Results presented in Example 3 above suggest that there may be unexpected improvements in efficacy attained by MDD-based ID delivery compared to that attained by Mantoux-based injections using standard needles. In addition, the MDD cannula mechanically disrupt a smaller total area of tissue since they are inserted to a reduced depth compared to standard needles and are not laterally “snaked” through the ID tissue like Mantoux-style injections. Tissue damage and inflammation leads to the release of several inflammatory proteins, chemokines, cytokines and other mediators of inflammation.

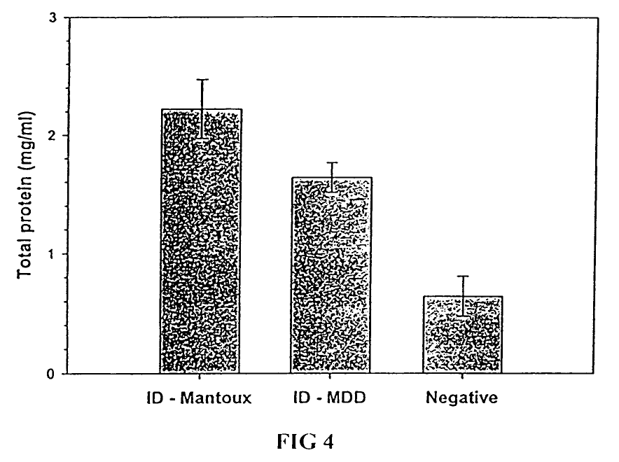

Thus, total protein content at recovered skin sites can be used as an indirect measurement of tissue damage and localized inflammation induced by the two delivery methods. Total protein levels were measured in recovered skin biopsies from pig samples presented in Example 3 above (excluding the 30 g, 1.5 mm) using a BCA assay (Pierce, Rockford, Ill.). Both methods of delivery induced an increase in total protein content compared to untreated skin, as shown in FIG. 4. However, total protein levels in recovered skin biopsies from the ID Mantoux group were significantly greater (p=0.01 by t-test) than the corresponding levels in the MDD group (2.4 mg/ml vs. 1.5 mg/ml). These results provide indirect evidence to strongly suggest that delivery by the methods of the present invention induces less mechanical damage to the tissue than the corresponding damage induced by Mantoux-style ID injection.

### Example 5

### Induction of Immune Response to Influenza DNA Vaccine Following ID Delivery in Rats

The examples presented above demonstrate that narrow gauge microcannula can be used to effectively deliver model nucleic acid based compounds into the skin resulting in high levels of gene expression by skin cells. To investigate the utility of delivering DNA vaccines by the methods of the present invention, rats were immunized with plasmid DNA encoding influenza virus hemagglutinin (HA) from strain A/PR/8/34 (plasmid provided by Dr. Harriet Robinson, Emory University School of Medicine, Atlanta, Ga.). Brown-Norway rats (n=3 per group) were immunized three times (days 0, 21 and 42) with plasmid DNA in PBS solution (50 μg per rat in 50 μl volume delivered by rapid bolus injection) ID using the MDD device as described in Example 2 or IM into the quadriceps using a conventional 30G needle and icc syringe. As a negative control, DNA was applied topically to untreated skin. Sera were collected at weeks 3, 5, 8 and 11 and analyzed for the presence of influenza-specific antibodies by ELISA. Briefly, microtiter wells (Nalge Nunc, Rochester, N.Y.) were coated with 0.1 μg of whole inactivated influenza virus (A/PR/8/34; Charles River SPAFAS, North Franklin, Conn.) overnight at 4° C. After blocking for 1 hr at 37° C. in PBS plus 5% skim milk, plates were incubated with serial dilutions of test sera for 1 hr at 37° C. Plates were then washed and further incubated with horse radish peroxidase conjugated anti-rat IgG, H+ L chain (Southern Biotech, Birmingham, Ala.) for 30 min at 37° C. and were then developed using TMB substrate (Sigma, St. Louis, Mo.). Absorbance measurements (A₄₅₀) were read on a Tecan Sunrise™ plate reader (Tecan, RTP, NC).

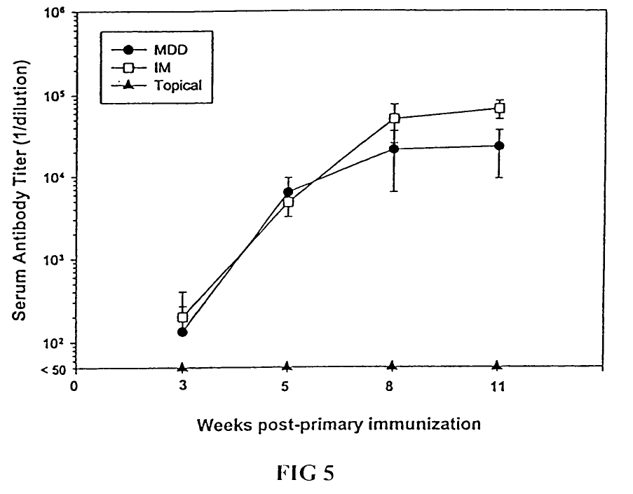

The results (FIG. 5) demonstrate that delivery by the method of the present invention of a genetic influenza vaccine in the absence of added adjuvant induces a potent influenza-specific serum antibody response. The magnitude of this response was comparable to that induced via IM injection at all measured timepoints. No detectable responses were observed in the topical controls. Thus delivery of genetic vaccine by the method of the present invention induces immune responses that are at least as strong as those induced by the conventional route of IM injection.

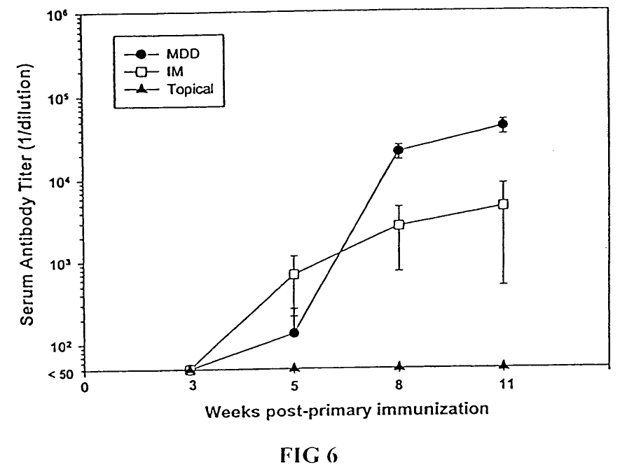

To further investigate delivery by the method of the present invention of adjuvanted genetic vaccines, the above described influenza HA-encoding plasmid DNA was prepared using the MPL+TDM Ribi adjuvant system (RIBI immunochemicals, Hamilton, Mont.) according to the manufacturer's instructions. Rats (n=3 per group) were immunized and analyzed for influenza-specific serum antibody as described above. Titers in the ID delivery group were comparable to IM following the first and second immunization (week 3-5; FIG. 6). After the third dose, however, ID-induced titers were significantly greater (p=0.03 by t-test) than the corresponding titers induced via IM injection (FIG. 6). At week 11, the mean ID-induced titer was 42,000 compared to only 4,600 attained by IM injection. Topical controls failed to generate an influenza-specific response. Thus, delivery by the method of the present invention of genetic vaccines in the presence of adjuvant induces immune responses that are stronger than those induced by the conventional route of IM injection.

### Example 6

### Induction of Immune Response to Influenza DNA/Virus “Prime-Boost” Following ID Delivery in Rats

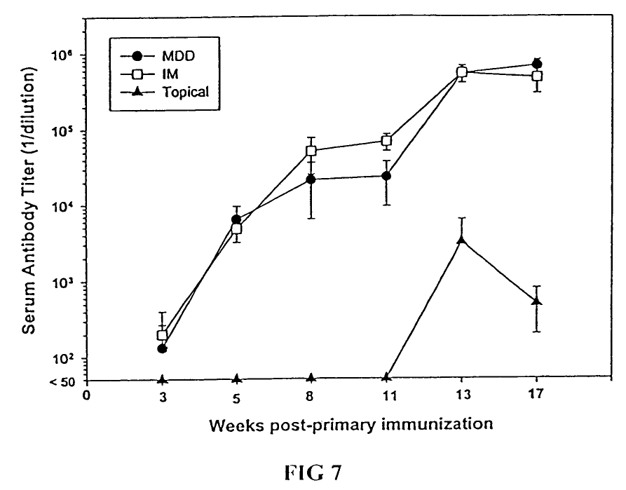

A recently developed vaccination approach for numerous diseases, including HIV, is the so-called “prime-boost” approach wherein the initial “priming” immunizations and secondary “boosters” employ different vaccine classes (Immunology Today, April 21(4): 163-165, 2000). For example, one may prime with a plasmid DNA version of the vaccine followed by a subsequent boost with a subunit protein, inactivated virus or vectored DNA preparation. To investigate delivery by the method of the present invention of these types of vaccination methods, the first experiment of Example 5 was continued for an additional 6 weeks. At week 11, DNA-primed rats were boosted with whole inactivated influenza virus (A/PR/8/34, 100 μg in 50 μl volume of PBS by rapid bolus injection). Virus was obtained from Charles River SPAFAS, North Franklin, Conn. Influenza-specific serum antibody titers were measured at weeks 13 and 17 by ELISA as described above. Both ID delivery and IM injection induced a potent booster effect (FIG. 7). Week 17 mean influenza-specific titers were equivalent (titer=540,000) by both methods of delivery and were significantly greater than the very weak titers observed following unassisted topical delivery (titer=3200). Thus, delivery by the method of the present invention is suitable for “prime-boost” immunization regimens, inducing immune responses that are at least as strong as those induced by the conventional route of IM injection.

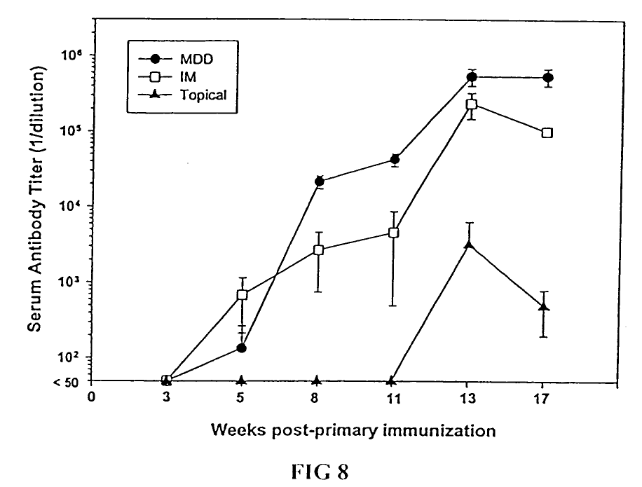

To evaluate the effect of adjuvant on the “prime-boost” response, the second experiment described in Example 5 was continued for an additional 6 weeks. At week 11, DNA-primed rats were boosted with whole inactivated influenza virus (A/PR/8/34, 100 μg in 50 μl volume by rapid bolus injection as above). Influenza-specific serum antibody titers were measured at weeks 13 and 17 by ELISA as described above. Both ID delivery and IM injection induced a potent booster effect (FIG. 8). Mean titers in the ID delivery group were greater than via IM injection following the virus boost; at week 13, the ID-MDD(MDD) mean titer was 540,000 compared to 240,000 by IM injection and 3,200 by unassisted topical application. Thus, delivery by the method of the present invention is suitable for “prime-boost” immunization regimens incorporating adjuvants, inducing immune responses that are stronger than those induced by the conventional route of IM injection.

### Example 7

### Induction of Immune Response to Influenza Virus Vaccine Following ID Delivery in Rats

To investigate the utility of delivering conventional vaccines by the method of the present invention in, rats were immunized with an inactivated influenza virus preparation as described in Example 6 via ID delivery or intra-muscular (IM) injection with a standard needle and syringe. As negative control, vaccine solution was applied topically to untreated skin; the large molecular weight of the inactivated influenza virus precludes effective immunization via passive topical absorption. As above, vaccine dose was 100 μg total protein in 50 μl volume per animal delivered by rapid bolus injection (<1 min). Rats were immunized 3 times (days 0, 21 and 42); serum was collected and analyzed for influenza-specific antibodies by ELISA as above on days 21, 35 and 56; n=4 rats per group.

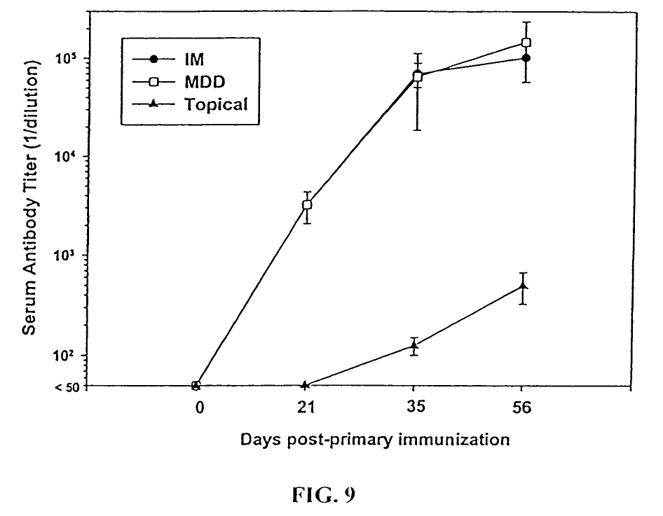

The results, shown in FIG. 9, indicate that ID delivery induces potent antigen specific immune responses. Similar levels of antibody were induced by the 2 injection routes, IM and ID. Peak geometric mean titers were somewhat higher in the ID-MDD group (MDD); 147,200 compared to 102,400 via IM injection. Topical application of the vaccine stimulated only very weak responses (peak mean titer=500). Thus, ID delivery of conventional vaccines at high doses induces immune responses that are at least as strong as those induced by the conventional route of IM injection.

### Example 8

### Induction of Immune Response to Influenza Vaccine Following ID Delivery Via in Pigs

As noted above, the pig represents an attractive skin model that closely mimics human skin. To test ID delivery devices in vaccine delivery, Yorkshire swine were immunized with an inactivated influenza vaccine as above, comparing ID delivery ID with IM injection. Pigs were immunized on days 0, 21 and 49; serum was collected and analyzed for influenza-specific antibodies by ELISA as above on days 14, 36, 49 and 60. Pig-specific secondary antibodies were obtained from Bethyl Laboratories, Montgomery, Tex.

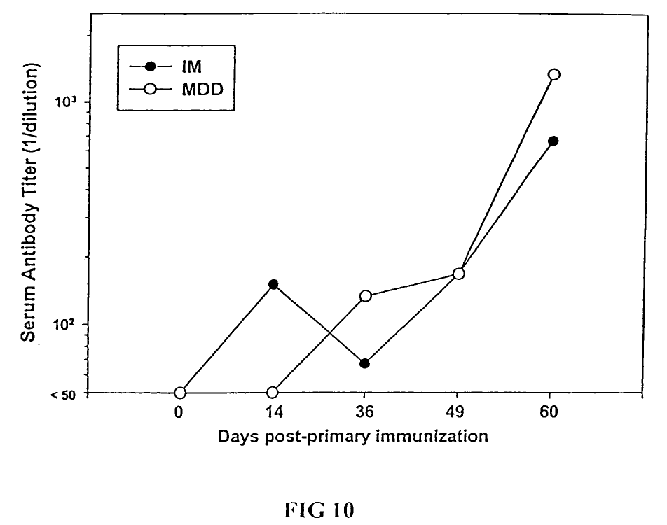

The results (FIG. 10) indicate that ID delivery induces potent antigen specific immune responses. Similar levels of antibody were induced by the 2 injection routes, IM and ID. Peak geometric mean titers were slightly higher in the MDD group; 1,333 compared to 667 via IM injection. Thus, ID delivery of conventional vaccines at high doses induces immune responses that are at least as strong as those induced by the conventional route of IM injection.

### Example 9

### ID Delivery of Lower Doses of Influenza Vaccine

In Example 7, rats were immunized with 100 μg of inactivated influenza virus via ID injection, or IM using a conventional needle and syringe. At such a high dose, both delivery methods induced similar levels of serum antibodies, although at the last time-point the ID-induced titer was slightly greater than that induced by IM.

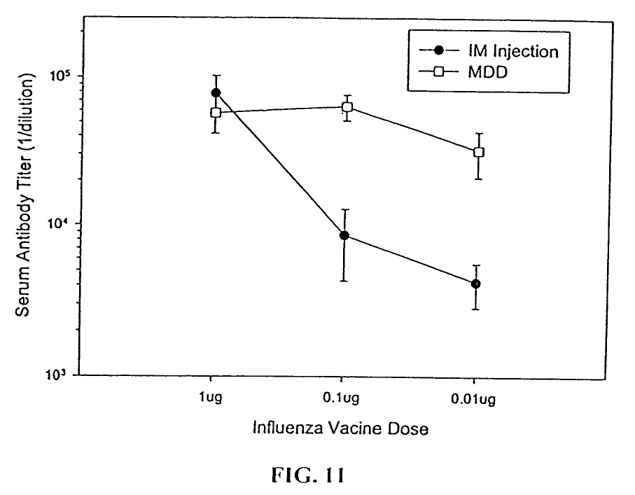

To further study the relationship between method of delivery and dosage level, rats were immunized with reduced doses of inactivated influenza virus, ranging from 1 μg to 0.01 μg per animal, using the ID and IM routes of administration as above. Rats were given 3 immunizations (days 0, 21 and 42) and were analyzed for serum anti-influenza antibodies at days 21, 35 and 56 (n=4 rats per group). Data shown in FIG. 11 reflect titers at day 56, although similar trends were observed at day 21 and day 35. ID delivery (MDD) resulted in a significant antibody response that did not differ significantly in magnitude at the 3 doses tested; i.e., delivery of as little as 0.01 μg (10 ng) induced comparable titers to those observed using 100-fold more vaccine (1 μg). In contrast, a significant reduction in titer was observed when the IM dose was reduced from 1 μg to 0.1 μg and again when the dose was further reduced to 0.01 μg. In addition, there was considerably less variability in the titers induced via ID delivery as compared to IM. Notably, no visible side reactions (adverse skin effects) were observed at the ID administration sites.

The results strongly indicate that ID delivery by the method of the present invention enables a significant (at least 100-fold) reduction in vaccine dose as compared to IM injection. Significant immune responses were observed using nanogram quantities of vaccine. Similar benefits would be expected in clinical settings.

The results described herein demonstrate that ID injection of vaccine and genetic material can be reproducibly accomplished the methods of the present invention. This method of delivery is easier to accomplish than standard Mantoux-style injections or IM and, in one embodiment, by virtue of its limited and controlled depth of penetration into the skin, is less invasive and painful. In addition, this method provides more reproducible ID delivery than via Mantoux style injections using conventional needles resulting in improved targeting of skin cells with resultant benefits as described above.

In addition, the method is compatible with whole-inactivated virus vaccine and with DNA plasmids without any associated reduction in biological potency as would occur if the virus particles or plasmid DNA were sheared or degraded when passed through the microcannula at the relatively high pressures associated with ID delivery in vivo. The results detailed herein demonstrate that stronger immune responses are induced via ID delivery, especially at reduced vaccine doses, thus potentially enabling significant dose reductions and combination vaccines in humans.

The results presented above show improved immunization via ID delivery using devices such as those described above as compared to standard intramuscular (IM) injection using a conventional needle and syringe. The dose reduction study (Example 9), shows that ID delivery induces serum antibody responses to an influenza vaccine in rats using at least 100-fold less vaccine than required via IM injection. If applicable in a clinical setting, such dose reduction would reduce or eliminate the problem of influenza vaccine shortages common across the world. In addition, such dose reduction capabilities may enable the delivery of a greater number of vaccine antigens in a single dose, thus enabling combination vaccines. This approach is of particular relevance to HIV vaccines where it likely will be necessary to immunize simultaneously with several genetic variants/sub-strains in order to induce protective immunity.

Similar benefits are expected with other types of prophylactic and therapeutic vaccines, immuno-therapeutics and cell-based entities by virtue of the improved targeting of the immune system using the methods and devices of the present invention.

In another embodiment, it is within the scope of the present invention to combine the ID delivery of the present invention with convention methods of administration, for example IP, IM, intranasal or other mucosal route, or SC injection, topical, or skin abrasion methods to provide improvement in immunological or therapeutic response. This would include for example, vaccines and or therapeutics of the same or different class, and administration simultaneously or sequentially.

All references cited in this specification are hereby incorporated by reference. The discussion of the references herein is intended merely to summarize the assertions made by their authors and no admission is made that any reference constitutes prior art relevant to patentability. Applicants reserve the right to challenge the accuracy and pertinence of the cited references.

1. The embodiments illustrated and discussed in the present specification are intended only to teach those skilled in the art the best way known to the inventors to make and use the invention, and should not be considered as limiting the scope of the present invention. The exemplified embodiments of the invention may be modified or varied, and elements added or omitted, without departing from the invention, as appreciated by those skilled in the art in light of the above teachings. It is therefore to be understood that, within the scope of the claims and their equivalents, the invention may be practiced otherwise than as specifically described.

What is claimed is:

1. A method for treating or preventing a disease in a subject comprising delivering to an intradermal compartment of the subject's skin, a vaccine comprising:
(a) a genetic material encoding an antigen that causes the disease; and/or
(b) an inactivated form of an antigen that causes the disease.

2. The method of claim 1 wherein said genetic material is a plasmid encoding an antigen, said antigen is a peptide or polypeptide.

3. The method of claim 1 wherein said antigen is a protein subunit, peptide, polypeptide or an inactivated virus.

4. The method of claim 1 wherein said disease is an infectious disease, genetic disorder or cancer.

5. The method of claim 1 wherein said antigen is influenza virus hemagglutinin.

6. The method of claim 1 wherein the amount of genetic material encoding an antigen that causes the disease is less than 0.5-1 μg, less than 1-2 μg, less than 2-4 μg, less than 4-10 μg, less than 10-20 μg, less than 20-40 μg, less than 40-60 μg, or less than 60-80 μg.

7. The method of claim 1 wherein the amount of an inactivated form of an antigen that causes the disease is less than 0.005-0.01 μg, less than 0.01-0.05 μg, less than 0.05-0.1 μg, less than 0.1-0.5 μg, or less than 0.5-0.8 μg.

8. A method for treating or preventing a disease in a subject comprising delivering to an intradermal compartment of the subject's skin:
(a) a vaccine comprising a genetic material encoding an antigen that causes the disease; and
(b) a vaccine comprising an inactivated form of an antigen that causes the disease.

9. The method of claim 8 wherein said genetic material is a plasmid encoding an antigen, said antigen is a peptide or polypeptide.

10. The method of claim 8 wherein said antigen is a protein subunit, peptide, polypeptide or an inactivated virus.

11. The method of claim 8 wherein said disease is an infectious disease, genetic disorder or cancer.

12. The method of claim 8 wherein said antigen is influenza virus hemagglutinin.

13. The method of claim 8 wherein the amount of genetic material encoding an antigen that causes the disease is less than 0.5-1 μg, less than 1-2 μg, less than 2-4 μg, less than 4-10 μg, less than 10-20 μg, less than 20-40 μg, less than 40-60 μg, or less than 60-80 μg.

14. The method of claim 8 wherein the amount of an inactivated form of an antigen that causes the disease is less than 0.005-0.01 g, less than 0.01-0.05 μg, less than 0.05-0.1 μg, less than 0.1-0.5 μg, or less than 0.5-0.8 μg

15. The method of claim 1 or 8 wherein steps (a) and (b) are performed sequentially.

16. A method for inducing an increased immune response in a subject comprising delivering a vaccine to an intradermal compartment of the subject's skin, wherein said immune response is higher than an immune response induced by delivery of the vaccine via an intramuscular route.

17. The method of claim 16 wherein said immune response is humoral and/or cellular immune response.

18. The method of claim 16 wherein said vaccine comprises a live, non-attenuated virus or viral vector.

19. The method of claim 16 wherein said vaccine comprises an inactivated or killed virus.

20. The method of claim 16 wherein said vaccine comprises a live, non-attenuated bacteria.

21. The method of claim 16 wherein said vaccine comprises an inactivated or killed bacterium.

22. The method of claim 16 wherein said vaccine comprises a nucleic acid.

23. The method of claim 16 wherein said vaccine additionally comprises a protein or peptide encoded by said nucleic acid.

24. The method of claim 16 wherein said vaccine further comprises an adjuvant.

25. The method of claim 16 wherein said vaccine comprises a polysaccharide or a polysaccharide-conjugate.
